(19) United States
(12) Patent Application Publication
(10) Pub. No.: US 2026/0098299 A1
(43) Pub. Date: Apr. 9, 2026

(54) GENE EXPRESSION-BASED IDENTIFICATION OF EARLY LYME DISEASE

(71) Applicants: The Regents of the University of California, Oakland, CA (US); The Johns Hopkins University, Baltimore, MD (US)

(72) Inventors: Charles Y. CHIU, San Francisco, CA (US); Jerome BOUQUET, San Francisco, CA (US); Venice SERVELLITA, San Francisco, CA (US); Mark J. SOLOSKI, Baltimore, MD (US); John N. AUCOTT, Lutherville, MD (US)

(73) Assignees: The Regents of the University of California, Oakland, CA (US); The Johns Hopkins University, Baltimore, MD (US)

(21) Appl. No.: 18/993,400

(22) PCT Filed: Jul. 12, 2023
(86) PCT No.: PCT/US2023/070083

§ 371 Date: Jan. 10, 2025

Related U.S. Application Data
(60) Provisional application No. 63/388,564, filed on Jul. 12, 2022.

(51) Int. Cl. C12Q 1/6883; A61K 31/43; A61K 31/545; A61K 31/546; A61K 31/65

(52) U.S. Cl. CPC C12Q 1/6883; A61K 31/43; A61K 31/545; A61K 31/546; A61K 31/65; C12Q 2600/158

(57) ABSTRACT

The present disclosure relates to measuring gene expression of cells of a blood sample obtained from a mammalian subject suspected of having a tick-borne disease. In particular, the present disclosure provides tools for determining whether a human subject has acute Lyme disease by transcriptome profiling a peripheral blood mononuclear cell or a whole blood sample from the subject.

### CROSS-REFERENCE TO RELATED APPLICATIONS

This application claims priority to and the benefit of U.S. Provisional Application No. 63/388,564, filed Jul. 12, 2022, the disclosure of which is incorporated herein by reference in its entirety.

### STATEMENT REGARDING FEDERALLY SPONSORED RESEARCH OR DEVELOPMENT

This invention was made with government support under W81XWH-17-1-0681 awarded by the Medical Research and Development Command, and R01 HL105704 and P30 AR053503 awarded by the National Institutes of Health. The government has certain rights in the invention.

### REFERENCE TO AN ELECTRONIC SEQUENCE LISTING

The content of the electronic sequence listing (643662002840SEQLIST.xml; Size: 55,975 bytes; and Date of Creation: Jul. 7, 2023) is herein incorporated by reference in its entirety.

### TECHNICAL FIELD

The present disclosure relates to measuring gene expression of cells of a blood sample obtained from a mammalian subject suspected of having a tick-borne disease. In particular, the present disclosure provides tools for determining whether a human subject has acute Lyme disease by transcriptome profiling a peripheral blood mononuclear cell sample or a whole blood sample from the subject.

### BACKGROUND

Lyme disease (also known as Lyme borreliosis) is a systemic disease caused by Borrelia burgdorferi, which is spread through bites of infected ticks. Lyme disease is the most common vector-borne disease in the United States, with nearly 500,000 Americans estimated from insurance records to be diagnosed and treated each year (see, e.g., CDC Lyme Disease Data and Statistics webpage; and Kugeler et al., Emerg Infect Dis, 27 (2): 616-619, 2021). If left undiagnosed and thus untreated, Lyme disease can cause arthritis, facial palsy, neuroborreliosis (neurological disease caused by B. burgdorferi that can include meningitis, radiculopathy, and occasionally encephalitis), and even myocarditis resulting in sudden death (see, e.g., CDC Lyme Disease Signs and Symptoms webpage). Most patients (80-90%) treated with appropriate antibiotics recover rapidly and completely, but a significant number of patients develop persistent or recurring symptoms. When treated patients develop prolonged symptoms, these patients are considered to have post-treatment Lyme disease syndrome (Aucott et al., Int J Infect Dis, 17: e443-e449). The length of recovery time from Lyme disease is linked to the timing of diagnosis and treatment. The longer Lyme disease remains undiagnosed and untreated, the longer recovery time will be (Margues, Infect Dis Clin North Am, 22:341-360, 2008).

Despite the advantages of early diagnosis and treatment, diagnosing Lyme disease at an early stage of disease development remains challenging (Branda et al., Clin Microbiol Rev, 34 (2): e00018-19, 2021). One reason for this is because clinical manifestations can be highly variable. Often, patients present with non-specific “flu-like” symptoms early in the course of the illness, and without a history of tick bite. The classic erythema migrans (EM) “bullseye” rash is seen in fewer than 70% of patients. The majority of individuals show either uniformly red skin lesions that can be mistaken for other skin conditions, or no skin lesions at all (Steere and Sikand, N Engl J Med, 348:2472-2474, 2003). Moreover, current diagnostic tests are only effective at a later stage of disease development or unable to reliably detect Lyme disease. The standard method is serological testing, and the CDC recommends a two-tier serological assay for Lyme disease diagnosis. Serological testing, however, misses the window of early acute infection and can be negative in up to 40% of early acute cases (Steere et al., Clin Infect Dis, 47:188-195, 2008). Another diagnostic option, nucleic acid testing, is hindered by low titers of B. burgdorferi in the blood during acute infection, and has a reported sensitivity of detection of only 20-62% (Aguero-Rosenfeld et al., Clin Microbiol Reg, 18:484-509, 2005; and Eshoo et al., PLOS One, 7: e36825, 2012). As such, clinicians from regions endemic for Lyme disease often make diagnoses on the basis of patient clinical presentation and history. Diagnoses based solely on clinical presentation result in some patients being inappropriately treated for Lyme disease, while other patients are not treated in a timely fashion. Ultimately, the failure to accurately diagnose Lyme disease due to the absence of a sensitive and specific test can lead to devastating outcomes, including sudden cardiac death from Lyme carditis (Forrester et al., MMWR, 63:982-983, 2014).

Thus, there exists a need for methods to specifically detect Lyme disease at the early acute stage in order to provide appropriate and timely treatment.

### SUMMARY

The present disclosure relates to measuring gene expression of cells of a blood sample obtained from a mammalian subject suspected of having a tick-borne disease. In particular, the present disclosure provides tools for determining whether a human subject has acute Lyme disease by transcriptome profiling a peripheral blood mononuclear cell sample or a whole blood sample from the subject.

As described in detail in Example 1, transcriptome profiling by RNA sequencing (RNA-Seq), targeted RNA-Seq, and/or machine learning-based classification was performed on 263 peripheral blood mononuclear cell samples from 218 subjects, including 94 early Lyme disease patients, 48 uninfected control subjects, and 57 patients with other infections (influenza, bacteremia, or tuberculosis). Differentially expressed genes among the 25,278 in the reference database were selected based on ≥1.5-fold change, ≤0.05 p-value, and ≤0.001 false discovery rate cutoffs. After gene selection using a k-nearest neighbor algorithm, the comparative performance of 10 different classifier models was evaluated using machine learning.

A 31-gene Lyme disease classifier (LDC) panel was identified that can discriminate between early Lyme patients and controls, with a subset of the 31 genes having previously been described in association with clinical investigations of Lyme disease patients or in vitro cell culture and rodent studies of Borrelia burgdorferi infection. Evaluation of the LDC using an independent test set of samples from 63 subjects yields an overall sensitivity of 90.0%, specificity of 100%, and accuracy of 95.2%. The LDC test is positive in 85.7% of seronegative patients and found to persist for ≥3 weeks in 9 of 12 (75%) patients. These results demonstrate the clinical utility of a gene expression classifier for diagnosis of early Lyme disease, including in patients negative by conventional serologic testing.

### BRIEF DESCRIPTION OF THE DRAWINGS

FIG. 1A shows a flowchart of the approach used to develop and validate a 31-gene classifier panel for identification of early Lyme disease. FIG. 1B shows a comparison of the performance (accuracy and kappa statistics) of ten different machine learning algorithms for Lyme disease classification based on training set data. Abbreviations: AUC-ROC (area under curve-receiver operating characteristic); CART (classification and regression tree); DEGs (differentially expressed genes); GLMNT (generalized linear model); KNN (k-nearest neighbor); LDA (linear discriminant analysis); ML (machine learning); NB (naïve bayes); NNET (neural network); PAM (partition around medoids); RF (random forest); SVML (linear support vector machine); SVMR (radial support vector machine); and TREx (targeted RNA expression sequencing).

FIG. 2A-FIG. 2D show results from a 31-gene Lyme disease classifier (LDC) derived using the generalized linear model machine learning algorithm. In this figure and associated experimental example, the disease score shown is a scaled Lyme score derived by scaling the raw Lyme score from 0.0 to 1.0 using the software package in R (see R-project website). The scaling was done for ease of visual representation with positive scores scaled to a value in a range greater than or equal to 0.3 and less than 1.0 (≥0.3 and <1.0=Lyme), and negative scores scaled to a value in a range greater than 0.0 and less than 0.3 (>0.0 and <0.3=non-Lyme). FIG. 2A shows a chart of misclassification error depending on the number of genes considered (upper x-axis) and related log (lambda) statistic (lower x-axis). FIG. 2B shows a receiver-operating-characteristic (ROC) curve of the performance of the LDC on a training set of 44 Lyme seropositive samples and 93 non-Lyme control samples, with an area under curve (AUC) of 0.972. The cutoff for positivity according to Youden's J statistic is 0.3. FIG. 2C shows violin plots of the LDC for an independent test set of 63 samples and for the training set of 137 samples. FIG. 2D shows 2×2 contingency tables of LDC test set performance overall and for serologically-confirmed seropositive and seronegative Lyme cases.

FIG. 3 shows a comparison of longitudinal testing between the LDC score and results from two-tiered Lyme serologic testing for Lyme seronegative and Lyme seropositive (both early and late seroconversion) patients at 0 and 3 weeks. Patients testing Lyme seropositive at 0 weeks did not get repeat serologic testing. CDC criteria for a positive Lyme serology include a positive screening ELISA and either ≥2 of 3 bands on reflex IgM testing (in patients with signs and symptoms lasting <30 days) or ≥5 of 10 bands on reflex IgG testing (Moore et al., Emerg Infect Dis, 22:1169-1177, 2016).

FIG. 4 shows a plot of the LDC score in 18 Lyme disease patients from available longitudinal samples at 0 weeks, 3 weeks, and 6 months. A Lyme disease classifier result is considered positive if the Lyme disease classifier score is greater than or equal to the 0.3 cutoff as determined using Youden's index (J statistic) from AUC-ROC. Patients are labeled P1 to P18.

FIG. 5 shows a flowchart of an exemplary method for determining whether a subject has or does not have Lyme disease. The Lyme disease score is the sum of the gene expression scores (read counts) for each of the genes of the Lyme classifier multiplied by their respective gene weights plus an intercept value.

### DETAILED DESCRIPTION

The present disclosure relates to measuring gene expression of cells of a blood sample obtained from a mammalian subject suspected of having a tick-borne disease. In particular, the present disclosure provides tools for determining whether a human subject has acute Lyme disease by transcriptome profiling a peripheral blood mononuclear cell sample or a whole blood sample from the subject.

Diagnosis of Lyme disease is often unreliable as it is typically made on the basis of tick exposure history and non-specific clinical findings. Erythema migrans, the “bull's-eye” rash associated with early Lyme disease, is seen less than 70% of patients and can be mistaken for other skin conditions and other diseases. For example, Southern tick associated rash illness (STARI), is also associated with the development of an erythematous bull's-eye rash around the tick bite, but is not caused by the Lyme agent (Borrelia burgdorferi in the United States) (Goddard, Am J Med, 130:231-233, 2017). Culture is impractical and rarely available, while serologic and nucleic acid testing for Borrelia have been of limited use due to low sensitivity. Moreover, Lyme disease serology often misses the window of early acute infection as patients present to the clinic prior to appearance of a detectable antibody response (Steere et al., Clin Infect Dis, 47:188-195, 2008).

Recent development of “omics” methods allow for the evaluation of novel diagnostic methods. The use of transcriptome profiling by next-generation sequencing (RNA-seq) is a promising approach to identify diagnostic host biomarkers in response to infection, such as tuberculosis (Anderson et al., N Eng J Med, 370:1712-1723, 2014), S. aureus bacteremia (Ahn et al., PLOS One, 8: e48979, 2013), or influenza (Woods et al., PLOS One, 8: e52198, 2013; and Zaas et al., Cell Host Microbe, 6:207-217, 2009). In the present disclosure, whole transcriptome sequencing and targeted RNA resequencing were used in conjunction with machine learning methods to define a panel of 31 human genes whose expression can distinguish samples obtained from acute Lyme disease patients from samples obtained from control subjects.

The Lyme disease classifier (LDC) provided in Table 1-5 showed a 90% sensitivity and 100% specificity in identifying clinical Lyme patients at time of initial presentation. A condensed diagnostic panel of 31 multiplexed gene targets is amenable to implementation on commercial multiplexed nucleic acid testing instruments (Poritz & Lingenfelter, “Multiplex PCR for Detection and Identification of Microbial Pathogens”, Advanced Techniques in Diagnostic Microbiology, 3^{rd} edition: Volume 2: Techniques (eds. Tang & Stratton): online resource XIV, Springer International Publishing: Imprint: Springer, Cham, 2018) or on targeted RNA next-generation sequencing platforms, with the latter being used in 2020-2021 for clinical SARS coronavirus 2 (SARS-COV-2) testing under FDA Emergency Use Authorization (First NGS-based COVID-19 diagnostic. Nat Biotechnol 38:777, 2020). During development of the present disclosure, 77% of Lyme disease patients with a positive LDC at initial presentation were found to remain positive for at least 3 weeks, consistent with earlier work on the Lyme disease transcriptome (Bouquet et al., mBio, e00100-00116, 2016). This observation indicates that an LDC classifier is useful for Lyme disease diagnosis during the approximately 3-week “window period” prior to generation of detectable antibody levels by two-tiered testing (Moore et al., Emerg Infect Dis, 22 (7): 1169-1177, 2016). Taken together, the LDC classifier meets 4 of the 5 characteristics of an “ideal” Lyme disease diagnostic (Schutzer et al., Clin Infect Dis, 68:1052-1057, 2019), including high sensitivity in early infection, high specificity, 24 hour or less turnaround time (if implemented on a multiplexed nucleic acid testing platform), and testing from easily collected samples such as blood. Thus, the LDC classifier may be useful as a complementary diagnostic to serologic testing, which exhibits high sensitivity (95-100%) in later stages of Lyme disease (the sole remaining characteristic out of 5), but inadequate sensitivity (29-77%) in early Lyme (Aguero-Rosenfeld et al., Clin Microbiol Rev, 18:484-509, 2005; and Branda et al., Clin Infect Dis, 66:1133-1139, 2018).

Some of the genes in the 31-gene LDC had previously been reported as related to Lyme disease based on in vitro and in vivo investigations. However, 7 of the 31 (25.8%) genes in the LDC had not been previously described in the literature (IGSF6, JMJD6, NIF3L1, SHCBP1, SYTL1, TTK and ZNF384). The identification of new differentially expressed genes associated with acute Lyme disease may be due in part to the inclusion of control samples from patients having acute febrile infections with viruses (e.g., influenza) or other bacteria (e.g., Mycobacterium tuberculosis).

Prior studies have used gene expression to profile Lyme disease patients from PBMCs (Bouquet et al., mBio, e00100-00116, 2016; Clarke et al., Front Immunol, 12:636289, 2021; and Petzke et al., mBio, 11, 2020), although the study of Example 1 incorporates larger numbers of Lyme disease cases and controls. The study by Clarke et al. (Front Immunol, 12:636289, 2021) reported development of a diagnostic classifier of 20 genes for early Lyme disease, but the performance was not evaluated with an independent test set. The study by Petzke et al. (mBio, 11, 2020) reported two kinds of classifiers for discriminating between Lyme disease cases and controls and between Lyme disease cases that resolve after treatment and those that progress to having persistent symptoms. All of these classifiers are limited by the absence of controls from other viral and bacterial infections to exclude overlapping immune and inflammatory response genes. In fact, only 2 genes in our LDC classifier, TYMS, a DNA replication and repair gene, and GRN, a cell proliferation gene, are shared with these prior classifiers (Clarke et al., Front Immunol, 12:636289, 2021; and Petzke et al., mBio, 11, 2020).

Other “omics” technologies have been used to develop classifiers for Lyme disease. For example, a previous study reported a metabolomic signature with 88% sensitivity and 95% specificity for identification of seropositive Lyme (Molins et al., Clin Infect Dis, 60:1767-1775, 2015), although the controls in that study were different (infectious mononucleosis, fibromyalgia, severe periodontitis, and syphilis). The methods of the present disclosure fared better, albeit tested on a smaller number of samples (220 samples compared to 461 samples). Thus, the full 31-gene Lyme disease classifier panel (ANPEP, ANXA5, ASPM, CASC5, CAV1, CDCA5, CXCL10, DRAM1, GBP4, GRN, IFI27, IFRD1, IGSF6, ITGAM, JMJD6, KIF2C, LDLR, MXD1, NIF3L1, PLK1, SHCBP1, SOCS3, SORT1, SPAG5, STAT1, SYTL1, TLR2, TPX2, TTK, TYMS and ZNF384) of the present disclosure is an important new tool for diagnosis of acute infection with Borrelia burgdorferi, especially during the early stages of infection, when IgM are not yet detectable, or in cases of seronegative Lyme disease (Rebman et al., Clin Rheumatol, 34:585-589, 2015; and Dattwyler et al., N Engl J Med, 319:1441-1446, 1988).

As ˜86% of samples from patients persistently seronegative at 0 and 3 weeks were correctly classified as Lyme, the LDC classifier of the present disclosure is contemplated to result in more accurate stratification of presumptive Lyme patients who have tested negative by serology. In the absence of “gold-standard” testing, it cannot be proven that these seronegative patients were infected by B. burgdorferi. Nevertheless, documentation of EM rash in all Lyme patients in this study, even in those who tested seronegative, concurrent “flu-like” symptoms, and enrollment during tick season in a region highly endemic for Lyme disease is highly suggestive of these individuals having acute Lyme disease. Evidence in support of infection is also provided by the finding that three of the four LDC-positive, seronegative patients exhibited borderline serologic responses just outside of formal CDC criteria for seropositivity. More accurate discrimination of Lyme patients using the LDC may be clinically useful by prompting diagnostic workup for a different tickborne disease or other acute illness. The identification of a subgroup of three patients (out of 10) with a persistently positive LDC signature at 6 months, two of whom had over 6 months of persistent symptoms, suggests that the LDC is also useful for diagnosis and monitoring of Lyme disease patients with chronic symptoms.

### I. Definitions

As used herein and in the appended claims, the singular forms “a,” “an” and “the” include plural referents unless otherwise indicated or clear from context. For example, “a polynucleotide” includes one or more polynucleotides.

It is understood that aspects and embodiments described herein as “comprising” include “consisting of” and “consisting essentially of” embodiments.

Reference to “about” a value or parameter describes variations of that value or parameter. For example, the term about when used in reference to 20% of ticks being suspected of being infected encompasses 18% to 22% of ticks being suspected of being infected.

The term “plurality” as used herein in reference to an object refers to three or more objects. For instance, “a plurality of genes” refers to three or more genes, preferably 3, 4, 5, 6, 7, 8, 9, 10, 11, 12, 13, 14, 15, 16, 17, 18, 19, 20, 25, 30, 35, 40, 45, or 50 more genes.

The term “portion” as used herein in reference to sequencing a member of an RNA expression library (e.g., mRNA or cDNA library) refers to determining the sequence of at least about 25, 50, 75, 100, 125, 150, 175, 200, 225, or 250 bases of the library member. In some embodiments, sequencing a portion may include sequencing the entire library member.

As used herein, the term “isolated” refers to an object (e.g., PBMC) that is removed from its natural environment (e.g., separated). “Isolated” objects are at least 50% free, preferably 75% free, more preferably at least 90% free, and most preferably at least 95% (e.g., 95%, 96%, 97%, 98%, or 99%) free from other components with which they are naturally associated.

As used herein, “a subject suspected of having a tick-borne disease” is a subject that meets one or more of the following criteria: has been bitten by a tick; has an erythema migrans rash; has flu-like symptoms (e.g., fatigue, fever, joint pain, and/or headaches); and has visited or resided in a region in which ticks are likely to be infected with a human pathogen (e.g., a bacterial, viral, or protozoal organism which is known to cause disease in infected humans).

As used herein, “early Lyme disease” refers to the acute stage of infection, when patients have had symptoms for less than or equal to 30 days.

The terms “treating” or “treatment” of a disease refer to executing a protocol, which may include administering one or more pharmaceutical compositions to an individual (human or other mammal), in an effort to alleviate signs or symptoms of the disease. Thus, “treating” or “treatment” does not require complete alleviation of signs or symptoms, does not require a cure, and specifically includes protocols that have only a palliative effect on the individual. As used herein, and as well-understood in the art, “treatment” is an approach for obtaining beneficial or desired results, including clinical results. Beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable.

As used herein, “AUC-ROC” refers to Area Under the Curve (AUC) of the Receiver Operating Characteristics (ROC) curve, and is used as a measure of model accuracy, ranging from 0 to 1, where 0 means that the model never predicts correctly (0% accuracy), and 1 means the model always predicts correctly (100% accuracy).

As used herein, the terms “Youden's index” and “J statistic” both refer to a single statistical metric that captures the performance of a dichotomous diagnostic test, in this case an LDC that discriminates between Lyme and non-Lyme. Youden's index, also equivalently referred to as J statistic or Youden's J statistic, integrates sensitivity and specificity information in its calculation, generating a value that ranges from 0 to 1, as follows: J statistic/Youden's Index=sensitivity+specificity−1. The point on AUC-ROC curve corresponding to the maximum Youden's index value corresponds to the sensitivity and specificity cutoffs that maximize the accuracy of a dichotomous diagnostic test.

### II. Methods for Measuring Gene Expression & Diagnosis of Acute Lyme Disease

Certain aspects of the present disclosure relate to methods for measuring gene expression, which may be used to assist in diagnosis of acute Lyme disease. In some embodiments, the methods include one or more techniques selected from of the group consisting of sequence analysis, hybridization, and amplification. For example, in some embodiments, the methods may include, without limitation, RT-qPCR, Luminex, Nanostring, and/or microarray. Exemplary methods are set forth below, but the skilled artisan will appreciate that various methods for measurement of gene expression that are known in the art can be employed without departing from the scope of the present disclosure.

In some embodiments, a method for measuring gene expression includes: (a) measuring RNA expression of a plurality of genes of peripheral blood mononuclear cells (PBMCs) isolated from a blood sample obtained from a mammalian subject suspected of having a tick-borne disease: (b) calculating a weighted RNA expression score for each of the plurality of genes; and (c) calculating a Lyme disease score by taking the sum of the weighted RNA expression scores. Thus, the gene expression of the plurality of genes forms the basis of the Lyme disease score used to diagnose acute Lyme disease. In some embodiments, the mammalian subject is a human. For example, in some embodiments, the Lyme disease score is the sum of the gene expression scores (read counts) for each of the genes of the Lyme classifier (plurality of genes) multiplied by their respective gene weights plus an intercept value (see Table 1-5). In some embodiments, the method further includes: step (d) identifying the subject as not having acute Lyme disease when the Lyme disease score is negative. In other embodiments, the method further includes: step (d) identifying the subject as having acute Lyme disease when the Lyme disease score is positive.

In some embodiments, the method further includes: obtaining a blood sample from the subject and isolating the PBMCs from the blood sample prior to step (a). The blood sample may be drawn into a container such as a cell preparation tube (CPT). For example, in some embodiments, the container used to collect the whole blood sample may include without limitation a BD Vacutainer® CPT™ Sodium Heparin or a BD Vacutainer® CPT™ EDTA. Subsequent to collection, PBMCs are isolated from the whole blood sample using a suitable cell separation method such as centrifugation through a polysaccharide density gradient medium (e.g., Ficoll-Paque® marketed by GE Healthcare, Lymphoprep® marketed by Alere Technologies AS, etc.).

In some embodiments, the method further includes: extracting RNA from the PBMCs prior to step (a). For example, in some embodiments, the method used to extract RNA may include, without limitation, Zymo Direct-Zol™, TRIzol® (reagents for isolating biological material marketed by Molecular Research Center, Inc.), phenol/chloroform, etc. RNA extraction may also include treating the RNA with DNAse to remove DNA contamination, which may occur during the extraction process (e.g., in an RNA extraction kit including an on-column DNAse step) or after the extraction process (e.g., DNAse treatment of extracted RNA). Subsequent to extraction, RNA concentration may be measured using a method such as Qubit fluorometric quantitation.

In some embodiments, the plurality of genes used in the method includes at least 3, 4, 5, 6 or all 7 genes of the group consisting of IGSF6, JMJD6, NIF3L1, SHCBP1, SYTL1, TTK and ZNF384. In some embodiments, the plurality of genes includes at least 3, 4, 5, 6, 7, 8, 9, 10, 11, 12, 13, 14, 15, 16, 17, 18, 19, 20, 21, 22, 23, 24, 25, 26 or all 27 genes of the group consisting of ANPEP, ASPM, CASC5, CAV1, CDCA5, CXCL10, DRAM1, GBP4, GRN, IFRD1, IGSF6, JMJD6, KIF2C, LDLR. MXD1, NIF3L1, SHCBP1, SOCS3, SORT1, SPAG5, STAT1, SYTL1, TLR2, TPX2, TTK, TYMS and ZNF384. In some embodiments, the plurality of genes does not include ANXA5, IFI27, ITGAM and/or PLK1. In some embodiments, the plurality of genes does not include C3orf14, CDCA2, CR1, GBP2, KCNJ2, KIF4A, MLF1IP, NCF1, PLBD1, RAD51, SLC25A37, STAB1, STEAP4, TBP, TNFSF13B, and/or ZNF276. In some embodiments, the plurality of genes does not include GRN and/or TYMS. In some embodiments, the plurality of genes does not include B4GALT1, CALU, HIST4H4, ICAM1, LYN, MMP9 and/or TPI1. In some embodiments, the plurality of genes used in the method includes at least 3, 4, 5, 6, 7, 8, 9, 10, 11, 12, 13, 14, 15, 16, 17, 18, 19, 20, 21, 22, 23, 24, 25, 26, 27, 28, 29, 30 or all 31 genes of the group consisting of ANPEP, ANXA5, ASPM, CASC5, CAV1, CDCA5, CXCL10, DRAM1, GBP4, GRN, IFI27, IFRD1, IGSF6, ITGAM, JMJD6, KIF2C, LDLR, MXD1, NIF3L1, PLK1, SHCBP1, SOCS3, SORT1, SPAG5, STAT1, SYTL1, TLR2, TPX2, TTK, TYMS and ZNF384. In some embodiments, the plurality of genes does not comprise C3orf14, CDCA2, CR1, GBP2, KCNJ2, KIF4A, MLF1IP, NCF1, PLBD1, RAD51, SLC25A37, STAB1, STEAP4, TBP, TNFSF13B, and/or ZNF276. In some embodiments, the plurality of genes consists of 86 genes or less, preferably 80 genes or less, preferably 70 genes or less, preferably 60 genes or less, preferably 50 genes or less, preferably 45 genes, preferably 40 genes or less, preferably 35 genes or less, or preferably 30 genes or less.

### A. Next Generation Sequencing Methods

In sequencing by synthesis, single-stranded DNA is sequenced using DNA polymerase to create a complementary second strand one base at a time. Most next generation (high-throughput) sequencing methods use a sequencing by synthesis approach, which is often combined with optical detection. High-throughput methods are advantageous in that many thousand (e.g., 106-109) sequences may be determined in parallel. Various high-throughput sequencing methods that may be used to measure gene expression in connection with the present disclosure are briefly described below.

Illumina (Solexa) sequencing, is a high-throughput method that uses reversible terminator bases for sequencing by synthesis (see e.g., Bentley et al., Nature, 456:53-59, 2008; and Meyer and Kircher, “Illumina Sequencing Library Preparation for Highly Multiplexed Target Capture and Sequencing”. Cold Springs Harbor Protocols 2010: doi: 10.1101/pdb.prot5448). First, DNA molecules are attached to a slide and amplified to generate local clusters of the same DNA sequence. Then, four types of fluorescently labeled nucleotides with reversible 3′ blockers (reversible terminator bases or RT-bases) are added to the chip, the excess is washed away, and the chip is imaged. After imaging, the dye and the 3′ blocker are removed from the nucleotide, and the next round of RT-bases is added to the chip and imaged.

Pyrosequencing is another type of sequencing by synthesis method that detects the release of pyrophosphate (PPi) during DNA synthesis (see, e.g., Ronaghi et al., Science, 281:363-365, 1998). In order to detect PPi, ATP sulfurylase, firefly luciferase, and luciferin are used, which together act to generate a visible light signal from PPi. Light is produced when a nucleotide has been incorporated into the complementary strand of DNA by DNA polymerase, and the intensity of the light emitted is used to determine how many nucleotides have been incorporated. Each of the four nucleotides is added in turn until the sequence is complete. High-throughput pyrosequencing, also known as 454 pyrosequencing (Roche Diagnostics), uses an initial step of emulsion PCR to generate oil droplets containing a cluster of single DNA sequences attached to a bead via primers. These droplets are then added to a plate with picoliter-volume wells such that each well contains a single bead as well as the enzymes needed for pyrosequencing.

Ion semiconductor sequencing (Ion Torrent, now Life Technologies) is a further type of sequencing by synthesis method that uses the hydrogen ions released during DNA polymerization for sequencing (see, e.g., U.S. Pat. No. 7,948,015). First, a single strand of template DNA is placed into a microwell. Then, the microwell is flooded with one type of nucleotide. If the nucleotide is complementary, it is incorporated into the secondary strand, and a hydrogen ion is released. The release of the hydrogen ion triggers a hypersensitive ion sensor; if multiple nucleotides are incorporated, multiple hydrogen ions are released, and the resulting electronic signal is higher.

Sequencing by ligation (SOLID sequencing marketed by Applied Biosystems) uses the mismatch sensitivity of DNA ligase in combination with a pool of fluorescently labeled oligonucleotides (probes) for sequencing (see, e.g., WO 2006084132). First, DNA molecules are amplified using emulsion PCR, which results in individual oil droplets containing one bead and a cluster of the same DNA sequence. Then, the beads are deposited on a glass slide. The probes are added to the slide along with a universal sequencing primer. If the probe is complementary, the DNA ligase joins it to the primer, fluorescence is measured, and then the fluorescent label is cleaved off. This leaves the 5′ end of the probe available for the next round of ligation.

Third-generation or long-read sequencing methods are high-throughput sequencing methods that sequence single molecules. These methods do not require initial PCR amplification steps. Single-molecule real-time sequencing (Pacific Biosciences) is a sequencing by synthesis long-read sequencing method, which employs zero-mode waveguides (ZMWs), which are small wells with capturing tools located at the bottom (see, e.g., Levene, Science, 299:682-686, 2003; and Eid et al., Science, 323:133-138, 2009). In brief, one DNA polymerase enzyme is attached to the bottom of a ZMW, and a single molecule of single-stranded DNA is present as a template. Four types of fluorescently-labelled nucleotides are present in a solution added to the ZMWs. When a nucleotide is incorporated into the second strand by the DNA polymerase in a ZMW, the fluorescence is detected by the capturing tools at the bottom of the ZMW. Then, the fluorescent label is cleaved off and diffuses away from the capturing tools at the bottom of the ZMW so it is no longer detectable and the remaining DNA strand in the ZMW is free of labels.

Nanopore sequencing (Oxford nanopore) is a sequencing method that sequences a single DNA or RNA molecule without any form of label. The principle of nanopore sequencing is that DNA passing through a nanopore changes the ion current of the nanopore in a manner dependent on the type of nucleotide. The nanopore itself contains a detection region able to recognize different nucleotides. Current nanopore sequencing methods in development are either solid state methods employing metal or metal alloys (see, e.g., Soni et al., Rev Sci Instrum, 81 (1): 014301, 2010) or biological employing proteins (see, e.g., Stoddartet al., Proc Natl Acad Sci USA, 106:7702-7707, 2009).

Further large-scale sequencing techniques for use in measuring gene expression in connection with methods of the present disclosure include but are not limited to microscopy-based techniques (e.g., using atomic force microscopy or transmission electron microscopy), tunneling currents DNA sequencing, sequencing by hybridization (e.g., using microarrays), sequencing with mass spectrometry (e.g., using matrix-assisted laser desorption ionization time-of-flight mass spectrometry, or MALDI-TOF MS), microfluidic Sanger sequencing, RNA polymerase (RNAP) sequencing (e.g., using polystyrene beads), and in vitro virus high-throughput sequencing.

Serial analysis of gene expression (SAGE) is a method that allows quantitative measurement of gene expression profiles that can be compared between samples (Velculescu et al., Science, 270:484-7, 1995). First, cDNA is synthesized from an RNA sample. Then, through multiple steps involving bead binding, cleavage, and adapters, short cDNA fragments (tags) are produced. These tags are concatenated, amplified using bacteria, isolated, and finally sequenced using high-throughput sequencing techniques. SAGE can be used to measure gene expression changes of multiple genes at once, for example in response to infection.

Specifically, in some embodiments of the present disclosure, measuring RNA expression of a plurality of genes includes targeted RNA expression resequencing including: (i) preparing an RNA expression library for the plurality of targeted genes from RNA extracted from the PBMCs; (ii) sequencing a portion of at least 50,000 members of the library; and (iii) generating a read count for RNA expression of the plurality of genes by normalization to the sequence of the at least 50,000 members of step (ii). In other embodiments, measuring RNA expression of a plurality of genes includes whole transcriptome shotgun sequencing (WTSS) including: (i) preparing an RNA expression library for the plurality of genes from RNA extracted from the PBMCs; (ii) sequencing a portion of at least 1,000,000 members of the library; and (iii) generating a read count for RNA expression of the plurality of genes by normalization to the sequence of the at least 1,000,000 members of step (ii). For example, library preparation may include, without limitation, the use of the Illumina TruSeq targeted RNA expression kit. The sequencing done in step (ii) of the above two embodiments may be, without limitation, Illumina MiSeq single-end reads 50 base pairs in length with a target sequencing depth of 200,000 reads per sample. The read count in step (iii) may be generated using any RNA library sequencing analysis methods (e.g., pipelines) known in the art. For example, these methods may include, without limitation, TopHat-Cufflinks, MiSeq reporter targeted RNA workflow, R software packages, graph-based analysis packages, and/or a combination thereof. In some embodiments, step (b) includes multiplying the read count for each of the plurality of genes by a predetermined gene expression weight to obtain the weighted RNA expression score (see Table 1-5). For example, in some embodiments, the predetermined gene expression weight may be calculated by an algorithm using additional information about the subject selected from the group containing age, sex, symptoms, time elapsed since tick bite, and/or previous Lyme disease diagnosis.

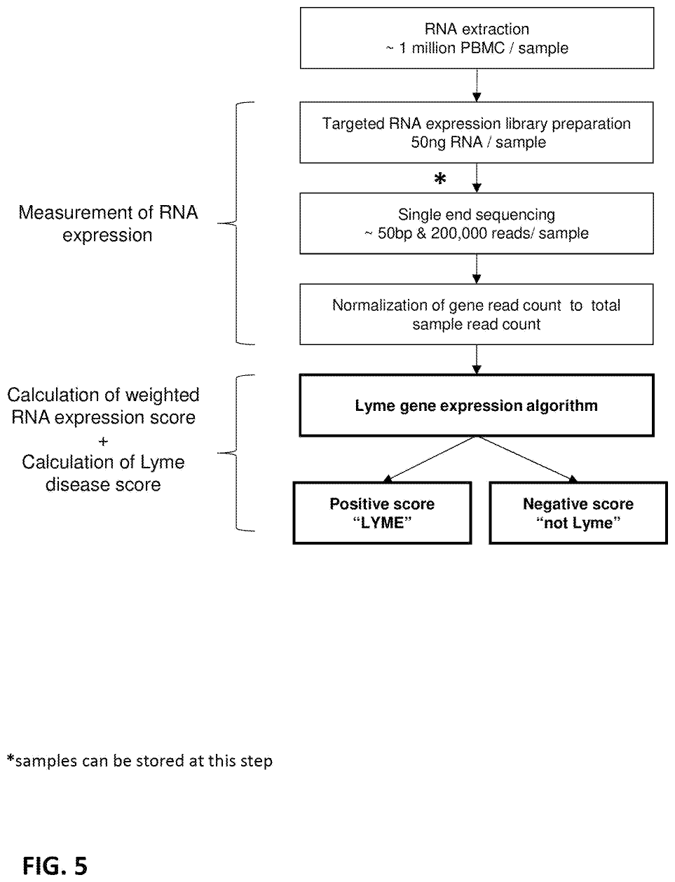

An exemplary method of measuring gene expression and diagnosing acute Lyme disease is illustrated in FIG. 5. As shown in FIG. 5, the process starts with RNA extraction from a sample containing about 1 million PBMCs. In the second step of the process, a targeted RNA expression library is prepared from a sample containing 50 ng of RNA. The expression library is targeted to a plurality of genes, as described above. After this second step, the samples can be stored for later processing. In the third step, the prepared library is sequenced using single end sequencing of about 50 base pairs, and a sequencing depth of 200,000 reads per sample. After the library is sequenced, the gene read count is normalized to the total sample read count in the fourth step. At the end of step four, the portion of the method used for RNA expression measurement (i.e. gene expression measurement) is complete. The fifth step is the first part of the portion of the method used for diagnosing acute Lyme disease. A Lyme gene expression algorithm is used to calculate the weighted RNA expression score. As described above, this Lyme gene expression algorithm may include additional information about the subject. In step six, the Lyme disease score is then calculated by taking the sum of the weighted RNA expression score. If the Lyme disease score is positive, the subject is diagnosed with Lyme disease, whereas if the Lyme disease score is negative, the subject is not diagnosed with Lyme disease.

### B. Amplification Methods for Measuring Gene Expression

Methods that may be used to measure gene expression in connection with the present disclosure may include an amplification step. In some embodiments of the present disclosure, measuring RNA expression of a plurality of genes includes a quantitative polymerase chain reaction (qPCR). For instance, some methods include performing reverse transcriptase-quantitative polymerase chain reaction (RT-qPCR) on RNA extracted from the PBMCs. Quantitative reverse transcription polymerase chain reaction (RT-qPCR) is an amplification method that uses fluorescence to quantitatively measure gene expression (see, e.g., Heid et al., Genome Res 6:986-994, 1996). The first step of RT-qPCR is to produce complementary DNA (cDNA) by reverse transcribing mRNA. The cDNA is used as the template in the PCR reaction. In addition to the template, gene-specific primers, a buffer (and other reagents for stability), a DNA polymerase, nucleotides, and a fluorophore are added to the PCR reaction. The reaction is then placed in a thermocycler that is able to both cycle through the different temperatures required for the standard PCR steps (e.g., separating the two strands of DNA, primer binding, and DNA polymerization) and illuminate the reaction with light at a particular wavelength to excite the fluorophore. Over the course of the reaction, the level of fluorescence is detected, and this level is subsequently used to quantify the amount of gene expression.

The use of fluorescence in RT-qPCR can be done in two different ways. The first way uses a dye in the reaction mixture that fluoresces when it binds to double stranded DNA. The intensity of the fluorescence increases as the amount of double stranded DNA increases, but the dye is not specific for a particular sequence. The second way uses sequence-specific probes labeled with a fluorescent reporter. The intensity of the fluorescence increases as the amount of the particular sequence increases.

RNA expression can also be quantified through reverse transcription-digital polymerase chain reaction (RT-dPCR). In this method the cDNA sample is prepared similarly to an RT-qPCR reaction, but the sample is diluted and divided into discrete subunits prior to amplification, such that each discrete subunit ideally contains either one or zero template cDNA molecules. Each subunit then undergoes PCR separately, and fluorescence can be used to detect the presence or absence of amplification of the target sequence in each subunit. Poisson statistics can then be used to extrapolate the quantification of the target sequence in the original sample, based on the ratio of subunits that contained the target sequence to subunits that did not contain the target sequence.

Amplification can also be performed without thermal cycling, using isothermal amplification methods. In these methods, DNA strands are separated using the strand displacement activity of certain DNA polymerases, including but not limited to Bst or Phi29 DNA polymerases. This allows for faster amplification of target sequences, and the ability to perform amplification at a constant temperature, thus eliminating the need for thermocycler equipment.

One method of isothermal amplification is loop-mediated isothermal amplification (LAMP), in which four to six primers are used to amplify a desired sequence by recognizing six to eight distinct regions of target DNA. Strand invasion by one primer allows a polymerase to separate the DNA strand, and complementarity of the primers allows for the formation of loops at the end of product DNA strand. This produces concatamers of product DNA which are readily detected, and can be quantified using fluorescent intercalators or probes and real-time fluorescence detection. LAMP products can also be measured using turbidity or dye to detect production of the by-product magnesium pyrophosphate.

Another method of isothermal amplification is recombinase polymerase amplification (RPA), in which the DNA strands are separated by the activity of a recombinase enzyme, allowing primers to invade into double-stranded DNA. In some embodiments, T4 UvsX is the recombinase and is used with its accessory protein UvsY. In some embodiments, the single-stranded binding protein gp32 is used to form D-loop recombination structures. In some embodiments, RPA is performed at about 37° C. In some embodiments, RPA is performed for clonal amplification in next generation sequencing workflows.

Further methods of isothermal amplification include nucleic acid sequence-based amplification (NASBA), rolling circle amplification (RCA), multiple displacement amplification (MDA), and whole genome amplification (WGA), which further comprises linear amplification via transposon insertion (LIANTI), multiple annealing and looping based amplification cycles (MALBAC), and degenerate oligonucleotide-primed PCR (DOP-PCR).

Thus, some methods of the present disclosure comprise performing a nucleic acid amplification technique (NAAT) on RNA extracted from the a cell sample, wherein the NAAT comprises a thermal cycle amplification technique or an isothermal amplification technique. In some embodiments, the methods comprise performing an isothermal amplification technique on RNA extracted from the cell sample. In some embodiments, RNA expression is measured using a technique selected from the group consisting of reverse-transcriptase-loop-mediated isothermal amplification (RT-LAMP), reverse-transcriptase-recombinase polymerase amplification (RT-RPA), reverse-transcriptase-nucleic acid sequence-based amplification (RT-NASBA), reverse transcription-strand displacement amplification (RT-SDA), reverse transcription-nicking enzyme amplification reaction (RT-NEAR), reverse transcriptase-helicase-dependent amplification (RT-HDA), reverse transcriptase-rolling circle amplification (RT-RCA), reverse-transcriptase-multiple displacement amplification (RT-MDA), and reverse-transcriptase-whole genome amplification (RT-WGA). In some embodiments, the isothermal amplification technique is selected from the group consisting of reverse transcriptase-loop-mediated isothermal amplification (RT-LAMP) and reverse transcriptase-recombinase polymerase amplification (RT-RPA).

### C. Hybridization Methods for Measuring Gene Expression

Methods that may be used to measure gene expression in connection with the present disclosure may include a hybridization step. In some preferred embodiments, the methods include use of a DNA microarray. DNA microarrays employ a plurality of specific DNA sequences (e.g., probes, reporters, oligos) attached to a slide or chip. First, cDNA from a sample is labeled with a fluorophore, silver, or a chemiluminescent molecule. Then, the labeled sample is hybridized to the DNA microarray under specific conditions, and hybridization is subsequently detected and quantified. Other methods of measuring gene expression through hybridization include but are not limited to Northern blot analysis, and in situ hybridization.

### III. Methods for Treating Lyme Disease

Certain aspects of the present disclosure relate to methods for treating Lyme disease. Exemplary methods of treatment are set forth below. Any of the methods for measuring gene expression described herein can be used for diagnosis or confirmation of acute Lyme disease in a subject in conjunction with treating Lyme disease. In some embodiments, treating Lyme disease includes administering an antibiotic therapy to the subject to treat the Lyme disease. In some embodiments, the antibiotic therapy includes an effective amount of an antibiotic selected from the group including: tetracyclines, penicillins, and cephalosporins. In other embodiments, the antibiotic therapy includes an effective amount of macrolides. In some embodiments, the antibiotic therapy includes an oral regimen including doxycycline, amoxicillin or cefuroxime axetil. In other embodiments, the antibiotic therapy includes a parenteral regimen including doxycycline, amoxicillin or cefuroxime axetil. In some embodiments, the antibiotic therapy includes an effective amount of doxycycline if the subject is an outpatient. In other embodiments, the antibiotic therapy includes an effective amount of ceftriaxone if the subject is hospitalized.

### IV. Kits for Measuring Gene Expression & Diagnosis of Acute Lyme Disease

Certain aspects of the present disclosure relate to kits for measuring gene expression and diagnosis of acute Lyme disease. In some embodiments, the kit includes: (a) a plurality of oligonucleotides which hybridize to a plurality of genes; and (b) instructions for: (i) use of the oligonucleotides for measuring RNA expression of the plurality of genes; (ii) calculating a weighted RNA expression score for each of the plurality of genes; and (iii) calculating a Lyme disease score by taking the sum of the weighted RNA expression scores.

In some embodiments, the plurality of genes used in the method includes at least 3, 4, 5, 6 or all 7 genes of the group consisting of IGSF6, JMJD6, NIF3L1, SHCBP1, SYTL1, TTK and ZNF384. In some embodiments, the plurality of genes includes at least 3, 4, 5, 6, 7, 8, 9, 10, 11, 12, 13, 14, 15, 16, 17, 18, 19, 20, 21, 22, 23, 24, 25, 26 or all 27 genes of the group consisting of ANPEP, ASPM, CASC5, CAV1, CDCA5, CXCL10, DRAM1, GBP4, GRN, IFRD1, IGSF6, JMJD6, KIF2C, LDLR, MXD1, NIF3L1, SHCBP1, SOCS3, SORT1, SPAG5, STAT1, SYTL1, TLR2, TPX2, TTK, TYMS and ZNF384. In some embodiments, the plurality of genes does not include ANXA5, IFI27, ITGAM and/or PLK1. In some embodiments, the plurality of genes does not include C3orf14, CDCA2, CR1, GBP2, KCNJ2, KIF4A, MLF1IP, NCF1, PLBD1, RAD51, SLC25A37, STAB1, STEAP4, TBP, TNFSF13B, and/or ZNF276. In some embodiments, the plurality of genes does not include GRN and/or TYMS. In some embodiments, the plurality of genes does not include B4GALT1, CALU, HIST4H4, ICAM1, LYN, MMP9 and/or TPI1. In some embodiments, the plurality of genes used in the method includes at least 3, 4, 5, 6, 7, 8, 9, 10, 11, 12, 13, 14, 15, 16, 17, 18, 19, 20, 21, 22, 23, 24, 25, 26, 27, 28, 39, 30 or all 31 genes of the group consisting of ANPEP, ANXA5, ASPM, CASC5, CAV1, CDCA5, CXCL10, DRAM1, GBP4, GRN, IFI27, IFRD1, IGSF6, ITGAM, JMJD6, KIF2C, LDLR, MXD1, NIF3L1, PLK1, SHCBP1, SOCS3, SORT1, SPAG5, STAT1, SYTL1, TLR2, TPX2, TTK, TYMS and ZNF384. In some embodiments, the plurality of genes does not comprise C3orf14, CDCA2, CR1, GBP2, KCNJ2, KIF4A, MLF1IP, NCF1, PLBD1, RAD51, SLC25A37, STAB1, STEAP4, TBP, TNFSF13B, and/or ZNF276. In some embodiments, the plurality of genes consists of 86 genes or less, preferably 80 genes or less, preferably 70 genes or less, preferably 60 genes or less, preferably 50 genes or less, preferably 45 genes, preferably 40 genes or less, preferably 35 genes or less, or preferably 30 genes or less.

In some embodiments, the plurality of oligonucleotides of the kit are attached to a slide or a chip. In some embodiments, the plurality of oligonucleotides of the kit each comprise a label for ease in detection. In some embodiments, the plurality of oligonucleotides comprise a pair of oligonucleotides for each of the plurality of genes. In some embodiments, the sequence of the pair of oligonucleotides is set forth in Table 1-1.

### Enumerated Embodiments

1. A method for measuring gene expression, comprising the steps of:
   (a) measuring RNA expression of a plurality of genes of cells from a blood sample obtained from a mammalian subject suspected of having a tick-borne disease;
   (b) calculating a weighted RNA expression score for each of the plurality of genes; and
   (c) calculating a Lyme disease score by taking the sum of the weighted RNA expression scores, wherein the plurality of genes comprises at least 3, 4, 5, 6 or all 7 genes of the group consisting of IGSF6, JMJD6, NIF3L1, SHCBP1, SYTL1, TTK and ZNF384.
2. The method of embodiment 1, wherein the plurality of genes further comprises at least 1, 2, 3, 4, 5, 6, 7, 8, 9, 10, 11, 12, 13, 14, 15, 16, 17, 18, 19 or all 20 genes of the group consisting of ANPEP, ASPM, CASC5, CAV1, CDCA5, CXCL10, DRAM1, GBP4, GRN, IFRD1, KIF2C, LDLR, MXD1, SOCS3, SORT1, SPAG5, STAT1, TLR2, TPX2 and TYMS.
3. The method of embodiment 2, wherein the plurality of genes further comprises at least 1, 2, 3 or all 4 of the group consisting of ANXA5, IFI27, ITGAM and PLK1.
4. The method of embodiment 1, wherein the plurality of genes comprises all 31 genes of the group consisting of ANPEP, ANXA5, ASPM, CASC5, CAV1, CDCA5, CXCL10, DRAM1, GBP4, GRN, IFI27, IFRD1, IGSF6, ITGAM, JMJD6, KIF2C, LDLR, MXD1, NIF3L1, PLK1, SHCBP1, SOCS3, SORT1, SPAG5, STAT1, SYTL1, TLR2, TPX2, TTK, TYMS and ZNF384.
5. The method of any one of embodiments 1-4, for providing information to assess whether a subject has early Lyme disease, further comprising: step (d) identifying the subject as not having early Lyme disease when the Lyme disease score is below a threshold value; or identifying the subject as having early Lyme disease when the Lyme disease score is above a threshold value.
6. The method of embodiment 5, wherein the threshold value is a maximum Youden's index value as plotted on an area under curve-receiver operating characteristic curve (AUC-ROC) that maximizes the accuracy of the LDC, optionally wherein the AUC-ROC is determined by use of a generalized linear model.
7. The method of any one of embodiments 1-6, further comprising: obtaining a blood sample from the subject prior to step (a).
8. The method of any one of embodiments 1-7, wherein the cells are peripheral blood mononuclear cells (PBMCs) isolated from the blood sample or wherein the cells are whole blood cells, optionally wherein the cells are peripheral blood mononuclear cells (PBMCs) isolated from the blood sample.
9. The method of any one of embodiments 1-8, further comprising: extracting RNA from the cells prior to step (a).
10. The method of any one of embodiments 1-9, wherein step (a) comprises one or more of the group consisting of sequence analysis, hybridization, and amplification.
11. The method of embodiment 10, wherein step (a) comprises targeted RNA expression resequencing comprising:
   (i) preparing an RNA expression library for the plurality of targeted genes from RNA extracted from the cells;
   (ii) sequencing a portion of at least 50,000 members of the library; and
   (iii) generating a read count for RNA expression of the plurality of genes by normalization to the sequence of the at least 50,000 members of step (ii).
12. The method of embodiment 10, wherein step (a) comprises whole transcriptome shotgun sequencing (WTSS) comprising:
   (i) preparing an RNA expression library for the plurality of genes from RNA extracted from the cells;
   (ii) sequencing a portion of at least 1,000,000 members of the library; and
   (iii) generating a read count for RNA expression of the plurality of genes by normalization to the sequence of the at least 1,000,000 members of step (ii).
13. The method of embodiment 11 or embodiment 12, wherein step (b) comprises: multiplying the read count for each of the plurality of genes by a predetermined gene expression weight to obtain the weighted RNA expression score.
14. The method of embodiment 10, wherein step (a) comprises: performing a nucleic acid amplification technique (NAAT) on RNA extracted from the PBMCs, wherein the NAAT comprises a thermal cycle amplification technique or an isothermal amplification technique.
15. The method of embodiment 14, wherein step (a) comprises performing a thermal cycle amplification technique on RNA extracted from the PBMCs, wherein the thermal cycle amplification technique is selected from the group consisting of reverse transcriptase-quantitative polymerase chain reaction (RT-qPCR) and reverse transcriptase-digital polymerase chain reaction (RT-dPCR).
16. The method of embodiment 14, wherein step (a) comprises performing an isothermal amplification technique on RNA extracted from the PBMCs, wherein the isothermal amplification technique is selected from the group consisting of reverse transcriptase-loop-mediated isothermal amplification (RT-LAMP) and reverse transcriptase-recombinase polymerase amplification (RT-RPA).
17. The method of embodiment 10, wherein step (a) comprises: hybridizing RNA extracted from the cells to a microarray.
18. The method of embodiment 10, wherein step (a) comprises: performing serial amplification of gene expression (SAGE) on RNA extracted from the cells.
19. The method of any one of embodiments 1-18, wherein the subject was bitten by a tick in a region where at least 20% of ticks are suspected of being infected with Borrelia burgdorferi.
20. The method of any one of embodiments 1-19, wherein the subject was bitten by a tick within three weeks of the blood sample being obtained.
21. The method of any one of embodiments 1-20, wherein the subject has an erythema migrans rash when the blood sample was obtained.
22. The method of any one of embodiments 1-20, wherein the subject does not have an erythema migrans rash when the blood sample was obtained.
23. The method of any one of embodiments 1-22, wherein the subject has flu-like symptoms when the blood sample was obtained.
24. The method of any one of embodiments 1-23, further comprising performing a serologic test for Lyme disease.
25. The method of embodiment 24, wherein the subject was determined to be negative for Lyme disease by serologic testing at the time the blood sample was obtained.
26. The method of any one of embodiments 1-25, wherein the tick-borne disease is selected from the group consisting of Borreliosis, Southern tick associated rash illness, Q fever, Colorado tick fever, Powassan virus infection, tick-borne encephalitis virus infection, tick-borne relapsing fever, Heartland virus infection and severe fever with thrombocytopenia virus infection.
27. The method of any one of embodiments 5-26, further comprising: step (e) administering an antibiotic therapy to the subject to treat the Lyme disease when the subject has been identified as having early Lyme disease.
28. The method of embodiment 27, wherein the antibiotic therapy comprises:
   (i) an effective amount of an antibiotic selected from the group consisting of tetracyclines, penicillins, and cephalosporins or
   (ii) an oral regimen comprising doxycycline, amoxicillin or cefuroxime axetil; or
   (iii) a parenteral regimen comprising ceftriaxone, cefotaxime, or penicillin G.
29. An antibiotic therapy for use in a method of treating Lyme disease in a subject that has been identified as having acute Lyme disease according to the method of any one of embodiments 5-26.
30. The antibiotic therapy for the use of embodiment 28, wherein the antibiotic therapy comprises:
   (i) an effective amount of an antibiotic selected from the group consisting of tetracyclines, penicillins, and cephalosporins or
   (ii) an oral regimen comprising doxycycline, amoxicillin or cefuroxime axetil; or
   (iii) a parenteral regimen comprising ceftriaxone, cefotaxime, or penicillin G.
31. A kit comprising:
   (a) a plurality of oligonucleotides which hybridize to a plurality of genes comprising at least 3, 4, 5, 6, or 7 genes of the group consisting of IGSF6, JMJD6, NIF3L1, SHCBP1, SYTL1, TTK and ZNF384;
   (b) instructions and/or an algorithm for: (i) use of the oligonucleotides for measuring RNA expression of the plurality of genes; (ii) calculating a weighted RNA expression score for each of the plurality of genes; and (iii) calculating a Lyme disease score by taking the sum of the weighted RNA expression scores.
32. The kit of embodiment 31, wherein the plurality of genes further comprises at least 1, 2, 3, 4, 5, 6, 7, 8, 9, 10, 11, 12, 13, 14, 15, 16, 17, 18, 19 or all 20 genes of the group consisting of ANPEP, ASPM, CASC5, CAV1, CDCA5, CXCL10, DRAM1, GBP4, GRN4, IFRD1, KIF2C, LDLR, MXD1, SOCS3, SORT1, SPAG5, STAT1, TLR2, TPX2 and TYMS.
33. The kit of embodiment 32, wherein the plurality of genes further comprises at least 1, 2, 3 or all 4 of the group consisting of ANXA5, IFI27, ITGAM and PLK1.
34. The kit of embodiment 31, wherein the plurality of genes comprises all 31 genes of the group consisting of ANPEP, ANXA5, ASPM, CASC5, CAV1, CDCA5, CXCL10, DRAM1, GBP4, GRN, IFI27, IFRD1, IGSF6, ITGAM, JMJD6, KIF2C, LDLR, MXD1, NIF3L1, PLK1, SHCBP1, SOCS3, SORT1, SPAG5, STAT1, SYTL1, TLR2, TPX2, TTK, TYMS and ZNF384.
35. An in vitro method for measuring RNA expression of a gene, comprising:
   i) obtaining RNA extracted from a peripheral blood mononuclear cell (PBMC) sample of a human subject suspected of having a tick-borne disease; and
   ii) performing targeted RNA expression resequencing comprising hybridization of an upstream oligonucleotide and a downstream oligonucleotide to the extracted RNA to measure RNA expression of the gene,
   wherein the gene comprises one or more of the group consisting of IGSF6, JMJD6, NIF3L1, SHCBP1, SYTL1, TTK and ZNF384.
36. The method of embodiment 35, wherein the gene comprises IGSF6 and the upstream oligonucleotide comprises the nucleotide sequence of SEQ ID NO:25 and the downstream oligonucleotide comprises the nucleotide sequence of SEQ ID NO:26.
37. The method of embodiment 35, wherein the gene comprises JMJD6 and the upstream oligonucleotide comprises the nucleotide sequence of SEQ ID NO:29 and the downstream oligonucleotide comprises the nucleotide sequence of SEQ ID NO:30.
38. The method of embodiment 35, wherein the gene comprises NIF3L1 and the upstream oligonucleotide comprises the nucleotide sequence of SEQ ID NO:37 and the downstream oligonucleotide comprises the nucleotide sequence of SEQ ID NO:38.
39. The method of embodiment 35, wherein the gene comprises SHCBP1 and the upstream oligonucleotide comprises the nucleotide sequence of SEQ ID NO:41 and the downstream oligonucleotide comprises the nucleotide sequence of SEQ ID NO:42.
40. The method of embodiment 35, wherein the gene comprises SYTL1 and the upstream oligonucleotide comprises the nucleotide sequence of SEQ ID NO:51 and the downstream oligonucleotide comprises the nucleotide sequence of SEQ ID NO:52.
41. The method of embodiment 35, wherein the gene comprises a plurality of genes comprises at least 3, 4, 5, 6 or all 7 of the group consisting of IGSF6, JMJD6, NIF3L1, SHCBP1, SYTL1, TTK and ZNF384, the upstream oligonucleotide comprises a plurality of upstream oligonucleotides, and the downstream oligonucleotide comprise a plurality of oligonucleotides, and
   wherein when the plurality of genes comprises:
   IGSF6, one of the plurality of upstream oligonucleotides comprises the nucleotide sequence of SEQ ID NO:25 and one of the plurality of downstream oligonucleotides comprises the nucleotide sequence of SEQ ID NO:26;
   JMJD6, one of the plurality of upstream oligonucleotides comprises the nucleotide sequence of SEQ ID NO:29 and one of the plurality of downstream oligonucleotides comprises the nucleotide sequence of SEQ ID NO:30;
   NIF3L1, one of the plurality of upstream oligonucleotides comprises the nucleotide sequence of SEQ ID NO:37 and one of the plurality of downstream oligonucleotides comprises the nucleotide sequence of SEQ ID NO:38;
   SHCBP1, one of the plurality of upstream oligonucleotides comprises the nucleotide sequence of SEQ ID NO:41 and one of the plurality of downstream oligonucleotides comprises the nucleotide sequence of SEQ ID NO:42;
   SYTL1, one of the plurality of upstream oligonucleotides comprises the nucleotide sequence of SEQ ID NO:51 and one of the plurality of downstream oligonucleotides comprises the nucleotide sequence of SEQ ID NO:52;
   TTK, one of the plurality of upstream oligonucleotides comprises the nucleotide sequence of SEQ ID NO:57 and one of the plurality of downstream oligonucleotides comprises the nucleotide sequence of SEQ ID NO:58; and/or
   ZNF384, one of the plurality of upstream oligonucleotides comprises the nucleotide sequence of SEQ ID NO:61 and one of the plurality of downstream oligonucleotides comprises the nucleotide sequence of SEQ ID NO:62.
42. The method of embodiment 41, wherein the plurality of genes comprises all 7 of the group consisting of IGSF6, JMJD6, NIF3L1, SHCBP1, SYTL1, TTK and ZNF384
43. The method of embodiment 42, wherein the plurality of genes further comprises at least 1, 2, 3, 4, 5, 6, 7, 8, 9, 10, 11, 12, 13, 14, 15, 16, 17, 18, 19 or all 20 of the group consisting of ANPEP, ASPM, CASC5, CAV1, CDCA5, CXCL10, DRAM1, GBP4, GRN, IFRD1, KIF2C, LDLR, MXD1, SOCS3, SORT1, SPAG5, STAT1, TLR2, TPX2 and TYMS, and
   wherein when the plurality of genes comprises:
   ANPEP, one of the plurality of upstream oligonucleotides comprises the nucleotide sequence of SEQ ID NO: 1 and one of the plurality of downstream oligonucleotides comprises the nucleotide sequence of SEQ ID NO:2;
   ASPM, one of the plurality of upstream oligonucleotides comprises the nucleotide sequence of SEQ ID NO:5 and one of the plurality of downstream oligonucleotides comprises the nucleotide sequence of SEQ ID NO:6;
   CASC5, one of the plurality of upstream oligonucleotides comprises the nucleotide sequence of SEQ ID NO:7 and one of the plurality of downstream oligonucleotides comprises the nucleotide sequence of SEQ ID NO:8;
   CAV1, one of the plurality of upstream oligonucleotides comprises the nucleotide sequence of SEQ ID NO:9 and one of the plurality of downstream oligonucleotides comprises the nucleotide sequence of SEQ ID NO:10;
   CDCA5, one of the plurality of upstream oligonucleotides comprises the nucleotide sequence of SEQ ID NO: 11 and one of the plurality of downstream oligonucleotides comprises the nucleotide sequence of SEQ ID NO:12;
   CXCL10, one of the plurality of upstream oligonucleotides comprises the nucleotide sequence of SEQ ID NO: 13 and one of the plurality of downstream oligonucleotides comprises the nucleotide sequence of SEQ ID NO:14;
   DRAM1, one of the plurality of upstream oligonucleotides comprises the nucleotide sequence of SEQ ID NO: 15 and one of the plurality of downstream oligonucleotides comprises the nucleotide sequence of SEQ ID NO:16;
   GBP4, one of the plurality of upstream oligonucleotides comprises the nucleotide sequence of SEQ ID NO:17 and one of the plurality of downstream oligonucleotides comprises the nucleotide sequence of SEQ ID NO:18;
   GRN, one of the plurality of upstream oligonucleotides comprises the nucleotide sequence of SEQ ID NO:19 and one of the plurality of downstream oligonucleotides comprises the nucleotide sequence of SEQ ID NO:20;
   IFRD1, one of the plurality of upstream oligonucleotides comprises the nucleotide sequence of SEQ ID NO:23 and one of the plurality of downstream oligonucleotides comprises the nucleotide sequence of SEQ ID NO:24;
   KIF2C, one of the plurality of upstream oligonucleotides comprises the nucleotide sequence of SEQ ID NO:31 and one of the plurality of downstream oligonucleotides comprises the nucleotide sequence of SEQ ID NO:32;
   LDLR, one of the plurality of upstream oligonucleotides comprises the nucleotide sequence of SEQ ID NO:33 and one of the plurality of downstream oligonucleotides comprises the nucleotide sequence of SEQ ID NO:34;
   MXD1, one of the plurality of upstream oligonucleotides comprises the nucleotide sequence of SEQ ID NO:35 and one of the plurality of downstream oligonucleotides comprises the nucleotide sequence of SEQ ID NO:36;
   SOCS3, one of the plurality of upstream oligonucleotides comprises the nucleotide sequence of SEQ ID NO:43 and one of the plurality of downstream oligonucleotides comprises the nucleotide sequence of SEQ ID NO:44;
   SORT1, one of the plurality of upstream oligonucleotides comprises the nucleotide sequence of SEQ ID NO:45 and one of the plurality of downstream oligonucleotides comprises the nucleotide sequence of SEQ ID NO:46;
   SPAG5, one of the plurality of upstream oligonucleotides comprises the nucleotide sequence of SEQ ID NO:47 and one of the plurality of downstream oligonucleotides comprises the nucleotide sequence of SEQ ID NO:48;
   STAT1, one of the plurality of upstream oligonucleotides comprises the nucleotide sequence of SEQ ID NO:49 and one of the plurality of downstream oligonucleotides comprises the nucleotide sequence of SEQ ID NO:50;
   TLR2, one of the plurality of upstream oligonucleotides comprises the nucleotide sequence of SEQ ID NO:53 and one of the plurality of downstream oligonucleotides comprises the nucleotide sequence of SEQ ID NO:54;
   TPX2, one of the plurality of upstream oligonucleotides comprises the nucleotide sequence of SEQ ID NO:55 and one of the plurality of downstream oligonucleotides comprises the nucleotide sequence of SEQ ID NO:56; and/or
   TYMS, one of the plurality of upstream oligonucleotides comprises the nucleotide sequence of SEQ ID NO:59 and one of the plurality of downstream oligonucleotides comprises the nucleotide sequence of SEQ ID NO:60.
44. The method of embodiment 43, wherein the plurality of genes further comprises all 20 of the group consisting of ANPEP, ASPM, CASC5, CAV1, CDCA5, CXCL10, DRAM1, GBP4, GRN, IFRD1, KIF2C, LDLR, MXD1, SOCS3, SORT1, SPAG5, STAT1, TLR2, TPX2 and TYMS.
45. The method of embodiment 44, wherein the plurality of genes further comprises at least 1, 2, 3 or all 4 of the group consisting of ANXA5, IFI27, ITGAM and PLK1, and wherein when the plurality of genes comprises:
   ANXA5, one of the plurality of upstream oligonucleotides comprises the nucleotide sequence of SEQ ID NO:3 and one of the plurality of downstream oligonucleotides comprises the nucleotide sequence of SEQ ID NO:4;
   IFI27, one of the plurality of upstream oligonucleotides comprises the nucleotide sequence of SEQ ID NO:21 and one of the plurality of downstream oligonucleotides comprises the nucleotide sequence of SEQ ID NO:22;
   ITGAM, one of the plurality of upstream oligonucleotides comprises the nucleotide sequence of SEQ ID NO:27 and one of the plurality of downstream oligonucleotides comprises the nucleotide sequence of SEQ ID NO:28 and/or
   PLK1, one of the plurality of upstream oligonucleotides comprises the nucleotide sequence of SEQ ID NO:39 and one of the plurality of downstream oligonucleotides comprises the nucleotide sequence of SEQ ID NO:40.
46. The method of embodiment 45, wherein the plurality of genes further comprises all 4 of the group consisting of ANXA5, IFI27, ITGAM and PLK1.
47. A kit comprising:
   (a) a plurality of upstream oligonucleotides and a plurality of downstream oligonucleotides, which hybridize to one of a plurality of genes comprising at least 3, 4, 5, 6, or 7 of the group consisting of IGSF6, JMJD6, NIF3L1, SHCBP1, SYTL1, TTK and ZNF384;
   (b) instructions for: (i) use of the oligonucleotides for measuring RNA expression of the plurality of genes by performing targeted RNA expression resequencing comprising hybridization of the plurality of upstream oligonucleotides and the plurality of a downstream oligonucleotides to RNA extracted from a peripheral blood mononuclear cell (PBMC) sample of a human subject suspected of having a tick-borne disease to measure RNA expression of the plurality of genes,
   wherein when the plurality of genes comprises:
   IGSF6, one of the plurality of upstream oligonucleotides comprises the nucleotide sequence of SEQ ID NO:25 and one of the plurality of downstream oligonucleotides comprises the nucleotide sequence of SEQ ID NO:26;
   JMJD6, one of the plurality of upstream oligonucleotides comprises the nucleotide sequence of SEQ ID NO:29 and one of the plurality of downstream oligonucleotides comprises the nucleotide sequence of SEQ ID NO:30;
   NIF3L1, one of the plurality of upstream oligonucleotides comprises the nucleotide sequence of SEQ ID NO:37 and one of the plurality of downstream oligonucleotides comprises the nucleotide sequence of SEQ ID NO:38;
   SHCBP1, one of the plurality of upstream oligonucleotides comprises the nucleotide sequence of SEQ ID NO:41 and one of the plurality of downstream oligonucleotides comprises the nucleotide sequence of SEQ ID NO:42;
   SYTL1, one of the plurality of upstream oligonucleotides comprises the nucleotide sequence of SEQ ID NO:51 and one of the plurality of downstream oligonucleotides comprises the nucleotide sequence of SEQ ID NO:52;
   TTK, one of the plurality of upstream oligonucleotides comprises the nucleotide sequence of SEQ ID NO:57 and one of the plurality of downstream oligonucleotides comprises the nucleotide sequence of SEQ ID NO:58; and/or
   ZNF384, one of the plurality of upstream oligonucleotides comprises the nucleotide sequence of SEQ ID NO:61 and one of the plurality of downstream oligonucleotides comprises the nucleotide sequence of SEQ ID NO:62.
48. The kit of embodiment 47, wherein the plurality of genes comprises all 7 of the group consisting of IGSF6, JMJD6, NIF3L1, SHCBP1, SYTL1, TTK and ZNF384.
49. The kit of embodiment 49, wherein the plurality of genes further comprises at least 1, 2, 3, 4, 5, 6, 7, 8, 9, 10, 11, 12, 13, 14, 15, 16, 17, 18, 19 or all 20 of the group consisting of ANPEP, ASPM, CASC5, CAV1, CDCA5, CXCL10, DRAM1, GBP4, GRN4, IFRD1, KIF2C, LDLR, MXD1, SOCS3, SORT1, SPAG5, STAT1, TLR2, TPX2 and TYMS, and
   wherein when the plurality of genes comprises:
   ANPEP, one of the plurality of upstream oligonucleotides comprises the nucleotide sequence of SEQ ID NO: 1 and one of the plurality of downstream oligonucleotides comprises the nucleotide sequence of SEQ ID NO:2;
   ASPM, one of the plurality of upstream oligonucleotides comprises the nucleotide sequence of SEQ ID NO:5 and one of the plurality of downstream oligonucleotides comprises the nucleotide sequence of SEQ ID NO:6;
   CASC5, one of the plurality of upstream oligonucleotides comprises the nucleotide sequence of SEQ ID NO:7 and one of the plurality of downstream oligonucleotides comprises the nucleotide sequence of SEQ ID NO:8;
   CAV1, one of the plurality of upstream oligonucleotides comprises the nucleotide sequence of SEQ ID NO:9 and one of the plurality of downstream oligonucleotides comprises the nucleotide sequence of SEQ ID NO:10;
   CDCA5, one of the plurality of upstream oligonucleotides comprises the nucleotide sequence of SEQ ID NO: 11 and one of the plurality of downstream oligonucleotides comprises the nucleotide sequence of SEQ ID NO:12;
   CXCL10, one of the plurality of upstream oligonucleotides comprises the nucleotide sequence of SEQ ID NO:13 and one of the plurality of downstream oligonucleotides comprises the nucleotide sequence of SEQ ID NO:14;
   DRAM1, one of the plurality of upstream oligonucleotides comprises the nucleotide sequence of SEQ ID NO:15 and one of the plurality of downstream oligonucleotides comprises the nucleotide sequence of SEQ ID NO:16;
   GBP4, one of the plurality of upstream oligonucleotides comprises the nucleotide sequence of SEQ ID NO:17 and one of the plurality of downstream oligonucleotides comprises the nucleotide sequence of SEQ ID NO:18;
   GRN, one of the plurality of upstream oligonucleotides comprises the nucleotide sequence of SEQ ID NO:19 and one of the plurality of downstream oligonucleotides comprises the nucleotide sequence of SEQ ID NO:20;
   IFRD1, one of the plurality of upstream oligonucleotides comprises the nucleotide sequence of SEQ ID NO:23 and one of the plurality of downstream oligonucleotides comprises the nucleotide sequence of SEQ ID NO:24;
   KIF2C, one of the plurality of upstream oligonucleotides comprises the nucleotide sequence of SEQ ID NO:31 and one of the plurality of downstream oligonucleotides comprises the nucleotide sequence of SEQ ID NO:32;
   LDLR, one of the plurality of upstream oligonucleotides comprises the nucleotide sequence of SEQ ID NO:33 and one of the plurality of downstream oligonucleotides comprises the nucleotide sequence of SEQ ID NO:34;
   MXD1, one of the plurality of upstream oligonucleotides comprises the nucleotide sequence of SEQ ID NO:35 and one of the plurality of downstream oligonucleotides comprises the nucleotide sequence of SEQ ID NO:36;
   SOCS3, one of the plurality of upstream oligonucleotides comprises the nucleotide sequence of SEQ ID NO:43 and one of the plurality of downstream oligonucleotides comprises the nucleotide sequence of SEQ ID NO:44;
   SORT1, one of the plurality of upstream oligonucleotides comprises the nucleotide sequence of SEQ ID NO:45 and one of the plurality of downstream oligonucleotides comprises the nucleotide sequence of SEQ ID NO:46;
   SPAG5, one of the plurality of upstream oligonucleotides comprises the nucleotide sequence of SEQ ID NO:47 and one of the plurality of downstream oligonucleotides comprises the nucleotide sequence of SEQ ID NO:48;
   STAT1, one of the plurality of upstream oligonucleotides comprises the nucleotide sequence of SEQ ID NO:49 and one of the plurality of downstream oligonucleotides comprises the nucleotide sequence of SEQ ID NO:50;
   TLR2, one of the plurality of upstream oligonucleotides comprises the nucleotide sequence of SEQ ID NO:53 and one of the plurality of downstream oligonucleotides comprises the nucleotide sequence of SEQ ID NO:54;
   TPX2, one of the plurality of upstream oligonucleotides comprises the nucleotide sequence of SEQ ID NO:55 and one of the plurality of downstream oligonucleotides comprises the nucleotide sequence of SEQ ID NO:56; and/or
   TYMS, one of the plurality of upstream oligonucleotides comprises the nucleotide sequence of SEQ ID NO:59 and one of the plurality of downstream oligonucleotides comprises the nucleotide sequence of SEQ ID NO:60.
50. The kit of embodiment 49, wherein the plurality of genes further comprises all 20 of the group consisting of ANPEP, ASPM, CASC5, CAV1, CDCA5, CXCL10, DRAM1, GBP4, GRN, IFRD1, KIF2C, LDLR, MXD1, SOCS3, SORT1, SPAG5, STAT1, TLR2, TPX2 and TYMS.
51. The kit of embodiment 50, wherein the plurality of genes further comprises at least 1, 2, 3 or all 4 of the group consisting of ANXA5, IFI27, ITGAM and PLK1, and
   wherein when the plurality of genes comprises:
   ANXA5, one of the plurality of upstream oligonucleotides comprises the nucleotide sequence of SEQ ID NO:3 and one of the plurality of downstream oligonucleotides comprises the nucleotide sequence of SEQ ID NO:4;
   IFI27, one of the plurality of upstream oligonucleotides comprises the nucleotide sequence of SEQ ID NO:21 and one of the plurality of downstream oligonucleotides comprises the nucleotide sequence of SEQ ID NO:22;
   ITGAM, one of the plurality of upstream oligonucleotides comprises the nucleotide sequence of SEQ ID NO:27 and one of the plurality of downstream oligonucleotides comprises the nucleotide sequence of SEQ ID NO:28 and/or
   PLK1, one of the plurality of upstream oligonucleotides comprises the nucleotide sequence of SEQ ID NO:39 and one of the plurality of downstream oligonucleotides comprises the nucleotide sequence of SEQ ID NO:40.
52. The kit of embodiment 51, wherein the plurality of genes further comprises all 4 of the group consisting of ANXA5, IFI27, ITGAM and PLK1.
53. The kit of any one of embodiments 47-52, further comprising:
   (c) an algorithm for calculating a weighted RNA expression score for each of the plurality of genes and calculating a Lyme disease score by taking the sum of the weighted RNA expression scores.

### EXAMPLES

In the experimental disclosure which follows, the following abbreviations apply: AUC (area under the curve); CART (classification and regression trees); DEG (differentially expressed gene); ELISA (enzyme-linked immunosorbent assay); EM (erythema migrans); FPKM (fragments per kilobase of exon per million fragments mapped); GLMNET (generalized linear model); KNN (k-nearest neighbor); KNNXV (k-nearest neighbor cross validation); LDA (linear discriminant analysis); LDC (Lyme disease classifier); NB (naïve bayes): NGS (next-generation sequencing): NNET (neural network); PAM (partition around medoids); PBMCs (peripheral blood mononuclear cells): RF (random forest); RPART (classification and regression tree); ROC (receiver-operating-characteristic curve); SVML (linear support vector machine); SVMR (radial support vector machine); TREx (targeted RNA expression sequencing); and WB (western blot).

### Example 1

### Gene Expression Classifier for the Early Detection of Lyme Disease

### Materials and Methods

All 94 Lyme disease subjects included in this study presented with a physician documented erythema migrans (EM) rash of ≥5 cm and either concurrent flu-like symptoms that included at least one of the following: fever, chills, fatigue, headache and/or new muscle or joint pains or dissemination of the EM rash to multiple skin locations. Controls (n=26) were enrolled from the same physician practice as cases. Two-tier serological Lyme disease testing was performed on clinical Lyme patients by a clinical reference laboratory (Quest Diagnostics) at the first visit and at 3 weeks, following a standard 3-week course of doxycycline treatment. Patients found to be Lyme seropositive at the first visit did not get repeat testing. Seropositivity was assessed according to established CDC criteria (Moore et al., Emerg Infect Dis, 22:1169-1177, 2016), including the requirement that patients have had symptoms for less than or equal to 30 days for Lyme diagnosis by positive ELISA and IgM testing. All controls were required to have a negative Lyme serologic test and no clinical history of Lyme disease to be enrolled into the study. All Lyme disease patients and controls were collected in Maryland, USA, an area highly endemic for Lyme disease.

PBMC samples from 57 patients diagnosed with other infections were collected at the University of California, San Francisco (UCSF) and 22 controls (asymptomatic blood donors) were collected at the Blood Systems Research Institute (BSRI) in San Francisco, California. Patients with other infections were diagnosed with either bacteremia (n=21), caused by Enterococcus faecium, Escherichia coli, Klebsiella pneumoniae, Staphylococcus aureus, Staphylococcus epidermidis, or Streptococcus pneumoniae by standard plate culture, or influenza (n=36) by positive RT-PCR testing (Luminex NxTAG Respiratory Pathogen Panel). PBMC samples from 19 adults, 9 patients diagnosed with tuberculosis using an interferon-gamma release assay (Oxford Immunotec T-SPOT.TB) and 10 uninfected controls, were collected at the British Columbia Centre for Disease Control (BCCDC) in Vancouver, Canada.

PBMCs were isolated from freshly collected whole blood in EDTA tubes (kept at 4° C. for <24 hours) using Ficoll (Ficoll-Paque Plus, GE Healthcare) and total RNA was extracted from 107 PBMCs using TRIzol reagent (Life Technologies). Messenger RNA (mRNA) was isolated from the total RNA using the Oligotex mRNA mini kit (Qiagen). The isolated mRNA was used to generate RNA-Seq libraries using the Scriptseq RNA-Seq library preparation kit (Epicentre) according to the manufacturer's protocol. The RNA-Seq libraries were then sequenced on a Hiseq 2000 instrument (Illumina).

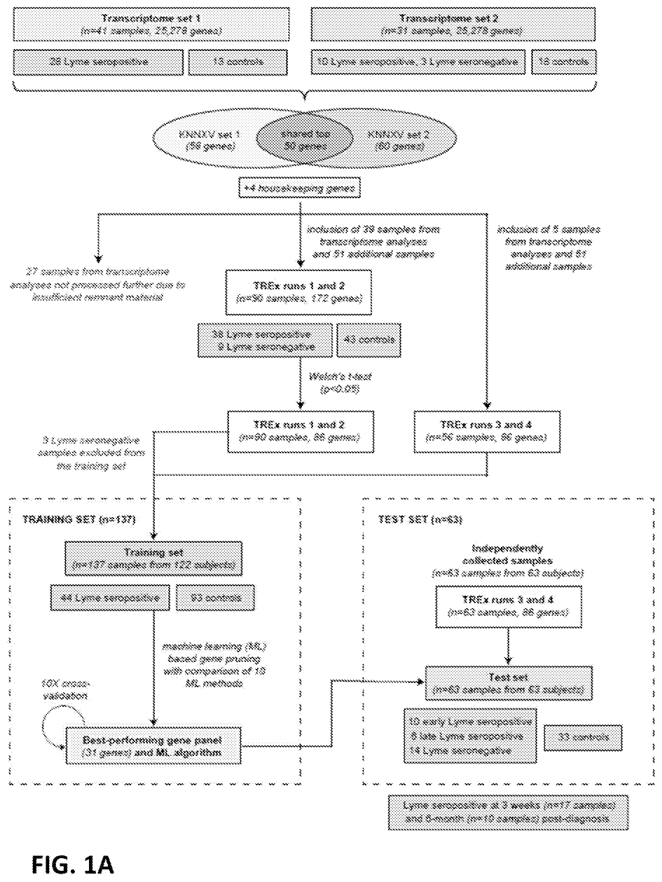

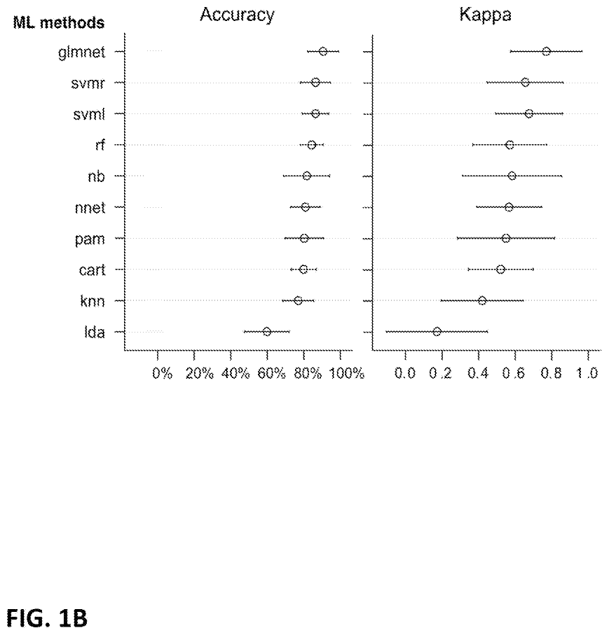

The samples were processed in two sets (FIG. 1). Set 1 corresponded to samples from 28 Lyme disease patients and 13 matched control patients (Bouquet et al., mBio 7, e00100-116, 2016). Set 2 corresponded to samples from 13 new Lyme disease patients and 6 matched control patients that were prepared and sequenced alongside samples from 6 influenza patients and 6 bacteremia patients. One sample was not included in the pooled analysis due to insufficient read counts.

Data analysis of the RNA-Seq library sequencing described above began by mapping the paired-end reads to the human genome (February 2009 human reference sequence [GRCh37/hg19] produced by the Genome Reference Consortium). After mapping, the exons were annotated and FPKM (fragments per kilobase of exon per million fragments mapped) values for all 25,278 expressed genes were calculated using version 2 of the TopHat-Cufflinks pipeline (Kim et al., Genome Biol, 14: R36, 2013). The differential expression of genes was calculated by using the ‘variance modeling at the observational level’ (voom) transformation (Law et al., Genome Biol, 15: R29, 2014), which applies precision weights to the matrix count, followed by linear modeling with the Limma package (Ritchie et al., Nucleic Acids Res, 43: e47, 2015). Genes were considered to be differentially expressed when the change was greater than or equal to 1.5-fold, the p-value was less than or equal to 0.05, and the adjusted p-value (or false discovery rate) was less than or equal to 0.1% (Dalman et al., BMC Bioinformatics, 13Suppl2: S11, 2012).

After the whole transcriptome analysis, a custom panel of transcripts of interest was selected for targeted RNA resequencing. The quantitative analysis of this custom panel was performed using a targeted RNA enrichment resequencing approach that used anchored multiplex PCR, and was done on a large number of samples. Here, PBMC samples (˜1 million cells) were extracted using Zymo Direct-zol™ RNA Miniprep Kit with on-column DNase following the manufacturer's instructions. Reverse transcription was performed on 50 ng of RNA following the manufacturer's instructions from the Illumina TruSeq Targeted RNA Expression Kit. Briefly, a custom panel of oligonucleotides (oligos), each capable of specifically hybridizing to one of the genes of interest, was designed and ordered using the Illumina DesignStudio platform. The oligos to genes of an exemplary 31 gene Lyme disease classifier panel are shown in Table 1-1. This pool of oligos, each attached to a small RNA sequencing primer (smRNA) binding site, was used to hybridize, extend and ligate the second strand of cDNA from our genes of interest. 35 cycles of amplification were then performed using primers with a complementary smRNA sequence, multiplexing index sequences, and sequencing adapters. The resulting libraries were sequenced on an Illumina MiSeq to a depth of ˜2,500 reads per sample per gene. Expression counts per sample per gene were performed on the instrument by MiSeq reporter targeted RNA workflow software (revision C). Briefly, following demultiplexing and FASTQ file generation, reads from each samples were aligned locally against references corresponding to targeted regions of interest using a banded Smith-Waterman algorithm (Okada et al., BMC Bioinformatics, 16:321, 2015). Normalization against the total number of reads from each sample and the machine learning algorithm were both done using R (see R-project website).

TABLE 1-1
Lyme Disease Classifier Oligonucleotides

| Gene Symbol | Upstream Locus Specific Oligo | Downstream Locus Specific Oligo |
|---|---|---|
| ANPEP | TTCTCCACCGAGTATGAGCTGCA | GAGCAGTTCAAGAAGGACAACGAGGAA |
| | (SEQ ID NO: 1) ANPEP | (SEQ ID NO: 2) ANPEP-rv |
| ANXAS | AGAATTTTGCCACCTCTCTTTATTCCA | GACTATAAGAAAGCTCTTCTGCTGCTC |
| | (SEQ ID NO: 3) ANXA5 | (SEQ ID NO: 4) ANXA5-rv |
| ASPM | GTGTGTCGCAGCGCTGTTTCGTGTT | TTTCTGTTAACTGGACACCACTCAA |
| | (SEQ ID NO: 5) ASPM | (SEQ ID NO: 6) ASPM-rv |
| CASC5 | CGCAAAATTTGTCGAGGAGGTTTGC | ATGGATGGGGTGTCTTCAGAGGCTAAT |
| | (SEQ ID NO: 7) CASC5 | (SEQ ID NO: 8) CASC5-rv |
| CAV1 | AACACCTCAACGATGACGTGGTCAA | ACTTTGAAGATGTGATTGCAGAACC |
| | (SEQ ID NO: 9) CAV1 | (SEQ ID NO: 10) CAV1-rv |
| CDCA5 | CCCGAGCATCCTCCCTGAAATCT | GTGCGGCTGCAGTCAGAAAGCCCAT |
| | (SEQ ID NO: 11) CDCA5 | (SEQ ID NO: 12) CDCA5-rv |
| CXCL10 | CTGCCTTATCTTTCTGACTCTAAGTGGC | GAACTGTACGCTGTACCTGCATCA |
| | (SEQ ID NO: 13) CXCL10 | (SEQ ID NO: 14) CXCL10-rv |
| DRAM1 | TTCTACTTCCTAACTTTCATCCAAGATTTC | ATCCACAGAAATCAATGGTGATATTTG |
| | (SEQ ID NO: 15) DRAM1 | (SEQ ID NO: 16) DRAM1-rv |
| GBP4 | GAGCATGAAAGGCTGCTAAAACACAAG | AAGAAATGCTTAAGGAAGAATTTCAAAAG |
| | (SEQ ID NO: 17) GBP4 | (SEQ ID NO: 18) GBP4-rv |
| GRN | AGGGACTTCCAGTTGCTGCCCCTT | GTGGCATGCGGGGATGGCCATCACT |
| | (SEQ ID NO: 19) GRN | (SEQ ID NO: 20) GRN-rv |
| IFI27 | TCAGCTTCACATTCTCAGGAACTCTC | TCTGGCTGAAGTTGAGGATCTCTTAC |
| | (SEQ ID NO: 21) IFI27 | (SEQ ID NO: 22) IFI27-rv |
| IFRD1 | TTGAAGGGATTAATTGACCTAACCCTGG | ACAAGGCAAGCAGCTCTTGAAGGTAT |
| | (SEQ ID NO: 23) IFRD1 | (SEQ ID NO: 24) IFRD1-rv |
| IGSF6 | ACCATAAGAGACATGTGGAAACAAATCAG | AGAAAGATAACAACACTTATGAAAACAGA |
| | (SEQ ID NO: 25) IGSF6 | (SEQ ID NO: 26) IGSF6-rv |
| ITGAM | GCCATGGCTCTCAGAGTCCTTCTGTTAA | GTTCAACTTGGACACTGAAAACGCA |
| | (SEQ ID NO: 27) ITGAM | (SEQ ID NO: 28) ITGAM-rv |
| JMJD6 | TACTACGAGAGCTTCTCGCTGAGC | TGGAAAGGGCAGATGCTTTACAGCT |
| | (SEQ ID NO: 29) JMJD6 | (SEQ ID NO: 30) JMJD6-rv |
| KIF2C | TTTCCCGGTCTCGCTATCAAGATCCAA | TAATTCACAGTGCCAATGTAAGGACTG |
| | (SEQ ID NO: 31) KIF2C | (SEQ ID NO: 32) KIF2C-rv |
| LDLR | GTGAGGGCTCTGTCCATTGTCCT | TTTGCCTGGGGGTCTTCCTTCTAT |
| | (SEQ ID NO: 33) LDLR | (SEQ ID NO: 34) LDLR-rv |
| MXD1 | AGGCGGCCGACTATCTGGAGCG | ATGGTTATGCCTCCATGTTACCATACA |
| | (SEQ ID NO: 35) MXD1 | (SEQ ID NO: 36) MXD1-rv |
| NIF3L1 | CCAGGGCGTCAACAACTGGTTGGCTAAA | GGCCCATACATCCTTCCAAAGCT |
| | (SEQ ID NO: 37) NIF3L1 | (SEQ ID NO: 38) NIF3L1-rv |
| PLK1 | GCAGCGTGCAGATCAACTTCTTC | ACACCAAGCTCATCTTGTGCCCA |
| | (SEQ ID NO: 39) PLK1 | (SEQ ID NO: 40) PLK1-rv |
| SHCBP1 | ACCTGCAAGAAAATGCTGAAGATGG | TAAAATTCAGACAAGTGGAGAGCCAGA |
| | (SEQ ID NO: 41) SHCBP1 | (SEQ ID NO: 42) SHCBP1-rv |
| SOCS3 | GCTGGACTGCTGCCACCCCGGGTGCT | GCCATAGCACTGATCAGTGACAATTTAC |
| | (SEQ ID NO: 43) SOCS3 | (SEQ ID NO: 44) SOCS3-rv |
| SORT1 | GTGCTCATTGTGAAGAAATATGTCTGTGG | CCTGGTGCATCGATACTCTGTGCT |
| | (SEQ ID NO: 45) SORT1 | (SEQ ID NO: 46) SORT1-rv |
| SPAG5 | AGAAAAACTAGATGACATTGTTCAGCA | AGAGGTGGTGAGGGGATGCAAAGAACT |
| | (SEQ ID NO: 47) SPAG5 | (SEQ ID NO: 48) SPAG5-rv |
| STAT1 | TGCCTTTGGAAAGTATTACTCCAGGC | ACCAGAGCCAATGGAACTTGATGG |
| | (SEQ ID NO: 49) STAT1 | (SEQ ID NO: 50) STAT1-rv |
| SYTL1 | TGTGAAAGAGAAGGAAGAGGGGCCAGA | ATGAGGCCCCTCAGGAGAGGCTCA |
| | (SEQ ID NO: 51) SYTL1 | (SEQ ID NO: 52) SYTL1-rv |
| TLR2 | ATTGCTCTTTCACTGCTTTCAACTGG | TTGAAGCACTGGACAATGCCACATAC |
| | (SEQ ID NO: 53) TLR2 | (SEQ ID NO: 54) TLR2-rv |
| TPX2 | AAGAGTTGCGGGAGGCTGTCGGCTAATAA | GCCAGACTTCAAGATTTCAGAAAAGGG |
| | (SEQ ID NO: 55) TPX2 | (SEQ ID NO: 56) TPX2-rv |
| TTK | ACTAAGCTTGAATAAAATTTCTGCTGATAC | TAACTCGGGAACTGTTAACCAAATTATG |
| | (SEQ ID NO: 57) TTK | (SEQ ID NO: 58) TTK-rv |
| TYMS | TATTTACCTGAATCACATCGAGCCACTG | CCAGACCTTTCCCAAAGCTCAGGATT |
| | (SEQ ID NO: 59) TYMS | (SEQ ID NO: 60) TYMS-rv |
| ZNF384 | GGAATAAGACGGCAGGAGGAAGAGAGA | ATGGAAGAATCTCACTTCAATTCTAACCCG |
| | (SEQ ID NO: 61) ZNF384 | (SEQ ID NO: 62) ZNF384-rv |

The k-nearest neighbor classification with leave-one-out cross validation algorithm (KNNXV) (Golub et al., Science, 286:531-537, 1999), as implemented on Genepattern (Reich et al., Nat Genet, 38:500-501, 2006), was used on the set of differentially expressed genes identified by RNA-Seq-based transcriptome profiling, using a k of three, signal to noise ratio feature selection, Euclidean distance, and by iteratively decreasing the number of features until reaching maximum accuracy.

Class prediction performance using a receiver-operator-characteristic (ROC) curve metric on targeted RNA resequencing read count results was tested using the glmnet (Friedman et al., J Stat Softw, 33:1-22, 2010) and caret (Kuhn, J Stat Softw, 28:1-26, 2008) packages in R software, version 3.01 (R Project for Statistical Computing) for 10 different machine learning methods at default parameters: classification and regression trees (‘rpart’ method) (Breiman et al., Classification and Regression Trees, Taylor & Francis, 1984), generalized linear models (‘glmnet’ method) (Friedman et al., J Stat Softw, 33:1-22, 2010), linear discriminant analysis (‘Ida’ method) (Ripley, Pattern Recognition and Neural Networks, Cambridge University Press, 1996), k-nearest neighbor (‘knn’ method) (Altman, Am Stat, 46:175-185, 1992), random forest (‘rf’ method) (Breiman, Mach Learn, 45:5-32, 2001), eXtreme Gradiant Boosting (‘xgbTree’ method) (Chen and Guestrin, KDD '16: Proceedings of the 22^{nd} ACM SIGKDD International Conference on Knowledge Discovery and Data Mining, 785-794, 2016), neural networks (‘nnet’ method) (Ripley, Pattern Recognition and Neural Networks, Cambridge University Press, 1996), linear and radial support vector machine (‘svmLinear’ and ‘svmRadial’ methods) (Suykens and Vandewalle, Neural Process Lett, 9:2930399, 1999), and nearest shrunken centroids (‘pam’ method) (Tibshirani et al., Proc Natl Acad Sci USA, 99:6567-6572, 2002). Subsequent feature selection and fitting of the glmnet or generalized linear models were performed using 10-fold cross-validation with regularization using lasso (least absolute shrinkage and selection operator) penalty and lambda (2) parameter. The value of lambda that provided the minimum mean cross-validated error was used to determine the optimal set of genes.

The performance of the classifier (KNNXV) was evaluated with the use of receiver-operating-characteristic (ROC) curves, calculation of area under the curve (AUC) (Hanley and McNeil, Radiobiology, 143:29-36, 1982), and estimates of sensitivity, specificity, negative predictive value, and positive predictive value.

The Mann-Whitney nonparametric test was used for the analysis of continuous variables, and Fisher's exact test was used for categorical variables. All confidence intervals were reported as two-sided binomial 95% confidence intervals. Statistical analysis was performed and plots were generated with R software, version 4.0.3 (R Project for Statistical Computing).

### Results

The study included samples from 220 subjects (Table 1-2). The 220 subjects included 90 Lyme disease patients, 55 infected “non-Lyme” controls with influenza (n=30), tuberculosis (n=10), and other bacteremia (n=15), and 75 uninfected asymptomatic controls. All Lyme patients, including 60 seropositive and 30 seronegative by clinical two-tiered antibody testing, had documented EM rash and history of tick exposure at the time of presentation, and were enrolled in the “Study of Lyme disease Immunology and Clinical Events” (SLICE) study at the Johns Hopkins Medical Institute. Control subjects categorized as uninfected asymptomatic were from regions with an incidence of Lyme disease of ≤0.2% (San Francisco, California and Vancouver, British Columbia) or had a negative Lyme serology test and no clinical history of tickborne disease. No significant differences in age or sex were noted between Lyme disease and control subjects.

TABLE 1-2
Demographic Of Patients With Early Lyme Disease And Healthy Controls

| Disease Cohort | Age | | | Positive Lyme | | P-value | P-value |
|---|---|---|---|---|---|---|---|
| & Location | (Avg/IQR/Range) | Females | | Serology¹ | | Age² | Sex³ |
| Lyme disease | 51 (42-64) [20-78] | 46/90 | (51.1%) | 60/90 | (66.7%) | 0.12 | 0.83 |
| MD, USA | | | | | | | |
| Uninfected | 53 (43-61) [22-73] | 38/75 | (50.7%) | 0/26 | (0%) * | 0.59 | 0.95 |
| MD & CA USA | | | | | | | |
| BC, Canada | | | | | | | |
| Tuberculosis | 54 (42-68) [22-76] | 3/10 | (30%) | ND | | 0.26 | 0.21 |
| BC, Canada | | | | | | | |
| Flu | 59 (36-82) [4-104] | 12/30 | (40%) | ND | | 0.08 | 0.75 |
| CA, USA | | | | | | | |
| Bacteremia | 59 (53-69) [23-81] | 7/15 | (46.7%) | ND | | 0.17 | 0.29 |
| CA, USA | | | | | | | |

¹Lyme serology unknown for healthy controls from regions not highly endemic for Lyme disease (British Columbia and California).
²Disease versus control age.
³Disease versus control sex.

Transcriptome profiling using RNA-Seq was initially performed on PBMC samples from 72 subjects, including 41 Lyme patients and 31 controls (FIG. 1). Included were 41 samples from 28 Lyme patients and 13 uninfected controls (set 1), as previously reported (Bouqet et al., mBio, 7: e00100-116, 2016). For the remaining 31 samples from 13 Lyme patients and 18 controls (set 2), a mean 30 (+17 SD) million reads were generated per sample. No batch effect based on geographic site of collection was observed. Differentially expressed genes were selected separately for each set of PBMC samples using the k-nearest neighbor classification with leave-one-out cross validation (KNNXV) ML feature selection algorithm (Golub et al., Science, 286:531-537, 1999). The best accuracy for sets 1 and 2 was achieved using a panel of 58 and 60 genes, respectively.

These genes, along with an additional top 50 DEGs that were ranked according to adjusted p-value/FDR in order of decreasing significance and did not overlap with the two panels, were then combined into a 172-gene targeted RNA sequencing panel (Table 1-3). The 172-gene panel was used to test 90 samples (38 Lyme seropositive, 9 Lyme seronegative, and 43 controls) over 2 targeted RNA expression sequencing runs (TREx, “targeted RNA expression” runs 1 and 2) (FIG. 1). A subset of 86 genes out of 172 (50%) with the maximum differences in gene expression between Lyme and “non-Lyme” control samples across the first 2 TREx runs was identified using Welch's t-test at a p<0.05 cutoff. The smaller 86-gene panel was then used to analyze an additional 119 samples in TREx runs 3 and 4.

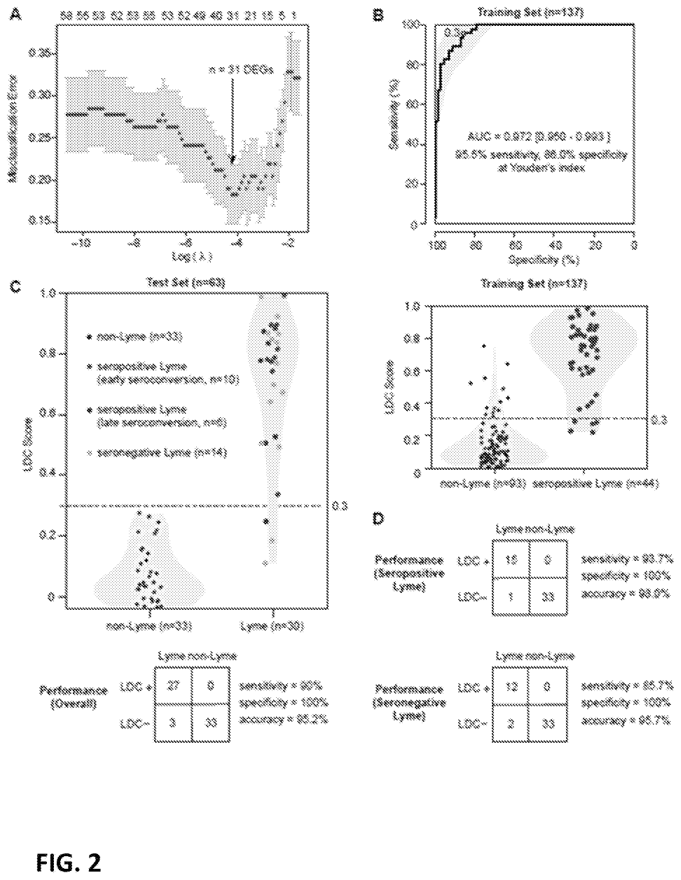

Next, ML-based methods were applied to select from the list of 86 candidate genes and determine the optimal combination of genes and classification model for the Lyme disease classifier (LDC), samples were randomly partitioned from TREx runs 1˜4 into a training set or test set. After ensuring that the training set consisted entirely of samples from laboratory-confirmed (“Lyme seropositive”) Lyme disease patients and that no prior analyses had been performed on the independent test set, 137 and 63 samples were assigned to the training and test sets, respectively, at an approximately 2:1 (68.5%: 31.5%) ratio (FIG. 1A). The training set was used to evaluate ten different machine learning algorithms for feature and model selection while varying the number of features (genes) from 1 to 86 for discriminating Lyme from non-Lyme patients using a 10-fold cross-validation scheme (FIG. 1B). A generalized linear model (“glmnet”) was found to provide the highest Area Under the Curve-Receiver Operating Characteristic (AUC-ROC) statistic (97.2%) with the AUC-ROC of other methods varying from 70-93%. The optimal cutoff as determined by Youden's J statistic (Youden, Cancer, 3:32-35, 1950) was 0.3. The highest AUC and lowest rate of misclassification error were found with a panel of 31 genes (FIG. 2A). This panel of 31 genes was then named the Lyme disease gene expression classifier (LDC), and was further tested using the validation set. Based on the expression of the 31 genes in the finalized Lyme disease classification panel (31 genes), a disease score ranging from 0.0 to 1.0 was calculated, with a score greater than or equal to 0.3 classified as Lyme and less than 0.3 classified as “non-Lyme”.

Compared to two-tier Lyme antibody testing as a reference gold standard, training set sensitivity, specificity, and AUC-ROC using this scoring metric were 95.5% (95% [84.1%-100%]), 86.0% (95% [77.4%-98.9%]), and 97.2 (95% [95.0%-99.3%]), respectively (FIG. 2B). Five of 44 (11.4%) Lyme samples and 12 of 93 controls (12.9%) in the training set were misclassified (FIG. 2C). Lyme disease gene expression classifier results between subjects who were seropositive at presentation had comparable sensitivity to those who were seropositive after 3 weeks (88% versus 89%, respectively). This panel of 31 genes was then named the Lyme disease gene expression classifier, and was further tested using the validation set.

The intercept value (−1.755) and gene weights of Table 1-5 were based on measurement of expression of the specific 31 genes of interest using targeted RNA sequencing. For this reason, if expression of fewer or more than 31 genes is measured, then the intercept value and gene weights may differ somewhat from the exemplary values. Similarly, if gene expression was measured using a different method, then the intercept value and gene weights may differ somewhat from the exemplary values. Targeted RNA sequencing results in infinite values expressed as read counts, which are dependent on the total sequencing depth. RT-qPCR on the other hand, results in finite values expressed in Ct (cycle threshold) in a range from 0 to 45. However, direction of the weight values (negative or positive) will remain the same, as they reflect which genes are under- and over-expressed in the context of Lyme disease.

TABLE 1-3
Targeted RNA Resequencing Assay Genes

| Gene symbol | GenBank No. | Gene name |
|---|---|---|
| ANXA5 | NM_001154 | Annexin A5 |
| ADAMTS10 | NM_030957 | A disintegrin and metalloproteinase with |
| | | thrombospondin motifs 10 |
| ALKBH2 | NM_001145375 | DNA oxidative demethylase ALKBH2 |
| ALPK1 | NM_025144 | Alpha-protein kinase 1 |
| ANPEP | NM_001150 | Aminopeptidase N |
| ARF4 | NM_001660 | ADP-ribosylation factor 4 |
| ARL5B | NM_178815 | ADP-ribosylation factor-like protein 5B |
| ASPM | NM_018136 | Abnormal spindle-like microcephaly-associated protein |
| AURKA | NM_198433 | Aurora kinase A |
| AZIN1 | NM_015878 | Antizyme inhibitor 1 |
| B4GALT5 | NM_004776 | Beta-1,4-galactosyltransferase 5 |
| BAZ1A | NM_013448 | Bromodomain adjacent to zinc finger domain protein 1A |
| BCL6 | NM_001706 | B-cell lymphoma 6 protein |
| BST1 | NM_004334 | ADP-ribosyl cyclase/cyclic ADP-ribose hydrolase 2 |
| BTNL8 | NM_024850 | Butyrophilin-like protein 8 |
| BUB1B | NM_001211 | Mitotic checkpoint serine/threonine-protein |
| | | kinase BUB1 beta |
| C16orf58 | NM_022744 | RUS1 family protein C16orf58 |
| C2orf89 | NM_001080824 | Metalloprotease TIKI1 |
| C3orf14 | NM_020685 | Uncharacterized protein C3orf14 |
| CASC5 | NM_170589 | Protein CASC5 |
| CASP1 | NM_033292 | Caspase-1 |
| CAV1 | NM_001753 | Caveolin-1 |
| CCDC130 | NM_030818 | Coiled-coil domain-containing protein 130 |
| CCL20 | NM_004591 | C-C motif chemokine 20 |
| CCNB1 | NM_031966 | G2/mitotic-specific cyclin-B1 |
| CCPG1 | NM_001204451 | Cell cycle progression protein 1 |
| CCR1 | NM_001295 | C-C chemokine receptor type 1 |
| CD300E | NM_181449 | CMRF35-like molecule 2 |
| CD3D | NM_000732 | T-cell surface glycoprotein CD3 delta chain |
| CD55 | NM_001114752 | Complement decay-accelerating factor |
| CDCA2 | NM_152562 | Cell division cycle-associated protein 2 |
| CDCA5 | NM_080668 | Sororin |
| CELF1 | NM_001172639 | CUGBP Elav-like family member 1 |
| CENPF | NM_016343 | Centromere protein F |
| CEP55 | NM_018131 | Centrosomal protein of 55 kDa |
| CKAP4 | NM_006825 | Cytoskeleton-associated protein 4 |
| CLU | NR_045494 | Clustered mitochondria protein homolog |
| CR1 | NM_000651 | Clusterin |
| CREB5 | NM_182898 | Complement receptor type 1 |
| CXCL10 | NM_001565 | Cyclic AMP-responsive element-binding protein 5 |
| CXCL9 | NM_002416 | C-X-C motif chemokine 10 |
| DEFA5 | NM_021010 | C-X-C motif chemokine 9 |
| DRAM1 | NM_018370 | Defensin-5 |
| DSE | NM_013352 | DNA damage-regulated autophagy modulator protein 1 |
| ECT2 | NM_018098 | Dermatan-sulfate epimerase |
| EIF2D | NM_006893 | Protein ECT2 |
| FABP5 | NM_001444 | Eukaryotic translation initiation factor 2D |
| FANCI | NM_001113378 | Fatty acid-binding protein, epidermal |
| FCAR | NM_133269 | Fanconi anemia group I protein |
| FCGR2A | NM_021642 | Immunoglobulin alpha Fc receptor |
| FDX1L | NM_001031734 | Low affinity immunoglobulin gamma Fc region |
| | | receptor II-a |
| FLT1 | NM_002019 | Adrenodoxin-like protein, mitochondrial |
| FPR2 | NM_001005738 | Vascular endothelial growth factor receptor 1 |
| GALT | NM_000155 | N-formyl peptide receptor 2 |
| GBP2 | NM_004120 | Galactose-1-phosphate uridylyltransferase |
| GBP4 | NM_052941 | Guanylate-binding protein 2 |
| GCA | NM_012198 | Guanylate-binding protein 4 |
| GGT3P | NR 003267 | Grancalcin |
| GLT1D1 | NM_144669 | Putative gamma-glutamyltranspeptidase 3 |
| GNG10 | NM_001198664 | Glycosyltransferase 1 domain-containing protein 1 |
| GNGS | NM_005274 | Guanine nucleotide-binding protein |
| | | G(I)/G(S)/G(O) gamma-10 |
| GPR15 | NM_005290 | Guanine nucleotide-binding protein |
| | | G(I)/G(S)/G(O) gamma-5 |
| GPX3 | NM_002084 | G-protein coupled receptor 15 |
| GRAP | NM_006613 | Glutathione peroxidase 3 |
| GRINA | NM_001009184 | GRB2-related adapter protein |
| GRN | NM_002087 | Protein lifeguard 1 |
| HAL | NM_002108 | Granulins |
| HBG2 | NM_000184 | Histidine ammonia-lyase |
| HCAR2 | NM_177551 | Hemoglobin subunit gamma-2 |
| HIST2H2BE | NM_003528 | Hydroxycarboxylic acid receptor 2 |
| HMBS | NM_001024382 | Histone H2B type 2-B |
| HSPA6 | NM_002155 | Porphobilinogen deaminase |
| ICAM1 | NM_000201 | Heat shock 70 kDa protein 6 |
| IFI27 | NM_005532 | Intercellular adhesion molecule 1 |
| IFRD1 | NM_001007245 | Interferon alpha-inducible protein 27, mitochondrial |
| IGSF6 | NM_005849 | Interferon-related developmental regulator 1 |
| IL23A | NM_016584 | Immunoglobulin superfamily member 6 |
| IL6 | NM_000600 | Interleukin-23 subunit alpha |
| ITGAM | NM_001145808 | Interleukin-6 |
| ITGB7 | NM_000889 | Integrin alpha-M |
| JMJD6 | NM_001081461 | Integrin beta-7 |
| KCNJ2 | NM_000891 | Bifunctional arginine demethylase and |
| | | lysyl-hydroxylase JMJD6 |
| KCNMB1 | NM_004137 | Inward rectifier potassium channel 2 |
| KIF2C | NM_006845 | Calcium-activated potassium channel subunit beta-1 |
| KIF4A | NM_012310 | Kinesin-like protein KIF2C |
| LDLR | NM_001195798 | Chromosome-associated kinesin KIF4A |
| LDOC1 | NM_012317 | Low-density lipoprotein receptor |
| LIMD2 | NM_030576 | Protein LDOC1 |
| LMNA | NM_170707 | LIM domain-containing protein 2 |
| LOC729737 | NR_039983 | Prelamin-A/C |
| LY9 | NM_002348 | T-lymphocyte surface antigen Ly-9 |
| MAP4K1 | NM_007181 | Mitogen-activated protein kinase kinase kinase kinase 1 |
| MBOAT2 | NM_138799 | Lysophospholipid acyltransferase 2 |
| MIR22HG | NR_028504 | Putative uncharacterized protein encoded by MIR22HG |
| MLF1IP | NM_024629 | Centromere protein U |
| MLLT6 | NM_005937 | Protein AF-17 |
| MSI2 | NM_138962 | RNA-binding protein Musashi homolog 2 |
| MXD1 | NM_002357 | Max dimerization protein 1 |
| MYBL2 | NM_002466 | Myb-related protein B |
| NANS | NM_018946 | Sialic acid synthase |
| NCF1 | NM_000265 | Neutrophil cytosol factor 1 |
| NIF3L1 | NM_021824 | NIF3-like protein 1 |
| NR3C2 | NM_000901 | Mineralocorticoid receptor |
| NUSAP1 | NM_018454 | Nucleolar and spindle-associated protein 1 |
| OAS2 | NM_016817 | 2′-5′-oligoadenylate synthase 2 |
| OMG | NM_002544 | Oligodendrocyte-myelin glycoprotein |
| ORC1 | NM_004153 | Origin recognition complex subunit 1 |
| OXSR1 | NM_005109 | Serine/threonine-protein kinase OSR1 |
| PABPC3 | NM_030979 | Polyadenylate-binding protein 3 |
| PECAM1 | NM_000442 | Platelet endothelial cell adhesion molecule |
| PHF15 | NM_015288 | Protein Jade-2 |
| PIK3R2 | NM_005027 | Phosphatidylinositol 3-kinase regulatory subunit beta |
| PKD1P1 | NR_036447 | Polycystin 1, transient receptor potential |
| | | channel interacting pseudogene 1 |
| PLBD1 | NM_024829 | Phospholipase B-like 1 |
| PLK1 | NM_005030 | Serine/threonine-protein kinase PLK1 |
| PNPLA1 | NM_173676 | Patatin-like phospholipase domain-containing protein 1 |
| POMT1 | NM_007171 | Protein O-mannosyl-transferase 1 |
| PSME1 | NM_006263 | Proteasome activator complex subunit 1 |
| QPCT | NM_012413 | Glutaminyl-peptide cyclotransferase |
| RAB12 | NM_001025300 | Ras-related protein Rab-12 |
| RAD51 | NM_133487 | DNA repair protein RAD51 homolog 1 |
| RBMX | NR_028477 | RNA-binding motif protein, X chromosome |
| RPL11 | NM_001199802 | 60S ribosomal protein L11 |
| RPL29 | NM_000992 | 60S ribosomal protein L29 |
| RPL6 | NM_001024662 | 60S ribosomal protein L6 |
| RPS5 | NM_001009 | 40S ribosomal protein S5 |
| RRM2 | NM_001165931 | Ribonucleoside-diphosphate reductase subunit M2 |
| SAMSN1 | NM_001256370 | SAM domain-containing protein SAMSN-1 |
| SERPINA1 | NM_001127705 | Alpha-1-antitrypsin |
| SERPING1 | NM_000062 | Plasma protease C1 inhibitor |
| SETD5 | NM_001080517 | SET domain-containing protein 5 |
| SHCBP1 | NM_024745 | SHC SH2 domain-binding protein 1 |
| SIGLEC5 | NM_003830 | Sialic acid-binding Ig-like lectin 5 |
| SIRPA | NM_080792 | Tyrosine-protein phosphatase non-receptor type substrate 1 |
| SIRPD | NM_178460 | Signal-regulatory protein delta |
| SLC15A3 | NM_016582 | Solute carrier family 15 member 3 |
| SLC25A37 | NM_016612 | Mitoferrin-1 |
| SLC31A2 | NM_001860 | Probable low affinity copper uptake protein 2 |
| SNRNP27 | NR_037862 | U4/U6.US small nuclear ribonucleoprotein 27 kDa protein |
| SOCS3 | NM_003955 | Suppressor of cytokine signaling 3 |
| SORT1 | NM_002959 | Sortilin |
| SPAG5 | NM_006461 | Sperm-associated antigen 5 |
| STAB1 | NM_015136 | Stabilin-1 |
| STAT1 | NM_007315 | Signal transducer and activator of transcription 1-alpha/beta |
| STEAP4 | NM_001205315 | Metalloreductase STEAP4 |
| STMN3 | NM_015894 | Stathmin-3 |
| SYTL1 | NM_032872 | Synaptotagmin-like protein 1 |
| TBCCD1 | NM_018138 | TBCC domain-containing protein 1 |
| TBP | NM_003194 | TATA-box-binding protein |
| TCEB1 | NM_001204861 | Transcription elongation factor B polypeptide 1 |
| TJP2 | NM_004817 | Tight junction protein ZO-2 |
| TLR2 | NM_003264 | Toll-like receptor 2 |
| TNFRSF10C | NM_003841 | Tumor necrosis factor receptor superfamily member 10C |
| TNFSF10 | NM_003810 | Tumor necrosis factor ligand superfamily member 10 |
| TNFSF13B | NM_006573 | Tumor necrosis factor ligand superfamily member 13B |
| TP53I13 | NM_138349 | Tumor protein p53-inducible protein 13 |
| TPM4 | NM_001145160 | Tropomyosin alpha-4 chain |
| TPX2 | NM_012112 | Targeting protein for Xklp2 |
| TREM1 | NM_018643 | Triggering receptor expressed on myeloid cells 1 |
| TTK | NM_003318 | Dual specificity protein kinase TTK |
| TXNDCS | NM_030810 | Thioredoxin domain-containing protein 5 |
| TYMP | NM_001953 | Thymidine phosphorylase |
| TYMS | NM_001071 | Thymidylate synthase |
| UBE2J1 | NM_016021 | Ubiquitin-conjugating enzyme E2 J1 |
| VASP | NM_003370 | Vasodilator-stimulated phosphoprotein |
| VMP1 | NM_030938 | Vacuole membrane protein 1 |
| WARS | NM_173701 | Tryptophan--tRNA ligase, cytoplasmic |
| WDR85 | NM_138778 | Diphthine methyltransferase |
| ZFP161 | NM_001243704 | Zinc finger and BTB domain-containing protein 14 |
| ZNF276 | NM_152287 | Zinc finger protein 276 |
| ZNF384 | NM_001135734 | Zinc finger protein 384 |
| ZNF549 | NM_001199295 | Zinc finger protein 549 |

TABLE 1-4
Lyme Disease Diagnostic Panel Genes

| Gene symbol | 1st gene panel | 2nd gene panel | 3rd gene panel |
|---|---|---|---|
| ANXA5 | yes | yes | yes |
| ADAMTS10 | yes | — | — |
| ALKBH2 | yes | — | — |
| ALPK1 | yes | — | — |
| ANPEP | yes | yes | yes |
| ARF4 | yes | — | — |
| ARL5B | yes | — | — |
| ASPM | yes | yes | yes |
| AURKA | yes | — | — |
| AZIN1 | yes | yes | — |
| B4GALT5 | yes | — | — |
| BAZ1A | yes | — | — |
| BCL6 | yes | — | — |
| BST1 | yes | yes | — |
| BTNL8 | yes | — | — |
| BUB1B | yes | yes | — |
| C16orf58 | yes | — | — |
| C2orf89 | yes | — | — |
| C3orf14 | yes | yes | — |
| CASC5 | yes | yes | yes |
| CASP1 | yes | yes | — |
| CAV1 | yes | yes | yes |
| CCDC130 | yes | yes | — |
| CCL20 | yes | — | — |
| CCNB1 | yes | yes | — |
| CCPG1 | yes | — | — |
| CCR1 | yes | — | — |
| CD300B | yes | — | — |
| CD3D | yes | yes | — |
| CD55 | yes | yes | — |
| CDCA2 | yes | yes | — |
| CDCA5 | yes | yes | yes |
| CELF1 | yes | — | — |
| CENPF | yes | yes | — |
| CEP55 | yes | yes | — |
| CKAP4 | yes | yes | — |
| CLU | yes | — | — |
| CR1 | yes | yes | — |
| CREB5 | yes | — | — |
| CXCL10 | yes | yes | yes |
| CXCL9 | yes | yes | — |
| DEFA5 | yes | yes | — |
| DRAM1 | yes | yes | yes |
| DSE | yes | — | — |
| ECT2 | yes | yes | — |
| EIF2D | yes | yes | — |
| FABPS | yes | yes | — |
| FANCI | yes | yes | — |
| FCAR | yes | — | — |
| FCGR2A | yes | — | — |
| FDX1L | yes | yes | — |
| FLT1 | yes | — | — |
| FPR2 | yes | yes | — |
| GALT | yes | — | — |
| GBP2 | yes | yes | — |
| GBP4 | yes | yes | yes |
| GCA | yes | — | — |
| GGT3P | yes | — | — |
| GLT1D1 | yes | — | — |
| GNG10 | yes | — | — |
| GNG5 | yes | — | — |
| GPR15 | yes | yes | — |
| GPX3 | yes | yes | — |
| GRAP | yes | — | — |
| GRINA | yes | — | — |
| GRN | yes | yes | yes |
| HAL | yes | — | — |
| HBG2 | yes | — | — |
| HCAR2 | yes | — | — |
| HIST2H2BE | yes | — | — |
| HMBS | yes | yes | — |
| HSPA6 | yes | — | — |
| ICAM1 | yes | yes | — |
| IFI27 | yes | yes | yes |
| IFRD1 | yes | yes | yes |
| IGSF6 | yes | yes | yes |
| IL23A | yes | — | — |
| IL6 | yes | — | — |
| ITGAM | yes | yes | yes |
| ITGB7 | yes | yes | — |
| JMJD6 | yes | yes | yes |
| KCNJ2 | yes | yes | — |
| KCNMB1 | yes | — | — |
| KIF2C | yes | yes | yes |
| KIF4A | yes | yes | — |
| LDLR | yes | yes | yes |
| LDOC1 | yes | — | — |
| LIMD2 | yes | — | — |
| LMNA | yes | yes | — |
| LOC729737 | yes | — | — |
| LY9 | yes | — | — |
| MAP4K1 | yes | — | — |
| MBOAT2 | yes | — | — |
| MIR22HG | yes | — | — |
| MLF1IP | yes | yes | — |
| MLLT6 | yes | — | — |
| MSI2 | yes | — | — |
| MXD1 | yes | yes | yes |
| MYBL2 | yes | yes | — |
| NANS | yes | — | — |
| NCF1 | yes | yes | — |
| NIF3L1 | yes | yes | yes |
| NR3C2 | yes | — | — |
| NUSAP1 | yes | yes | — |
| OAS2 | yes | yes | — |
| OMG | yes | — | — |
| ORC1 | yes | yes | — |
| OXSR1 | yes | — | — |
| PABPC3 | yes | — | — |
| PECAM1 | yes | — | — |
| PHF15 | yes | — | — |
| PIK3R2 | yes | — | — |
| PKD1P1 | yes | — | — |
| PLBD1 | yes | yes | — |
| PLK1 | yes | yes | yes |
| PNPLA1 | yes | — | — |
| POMT1 | yes | yes | — |
| PSME1 | yes | yes | — |
| QPCT | yes | — | — |
| RAB12 | yes | yes | — |
| RAD51 | yes | yes | — |
| RBMX | yes | — | — |
| RPL11 | yes | — | — |
| RPL29 | yes | — | — |
| RPL6 | yes | — | — |
| RPSS | yes | — | — |
| RRM2 | yes | yes | — |
| SAMSN1 | yes | — | — |
| SERPINA1 | yes | — | — |
| SERPING1 | yes | — | — |
| SETD5 | yes | — | — |
| SHCBP1 | yes | yes | yes |
| SIGLEC5 | yes | — | — |
| SIRPA | yes | — | — |
| SIRPD | yes | yes | — |
| SLC15A3 | yes | — | — |
| SLC25A37 | yes | yes | — |
| SLC31A2 | yes | — | — |
| SNRNP27 | yes | — | — |
| SOCS3 | yes | yes | yes |
| SORT1 | yes | yes | yes |
| SPAG5 | yes | yes | yes |
| STAB1 | yes | yes | — |
| STAT1 | yes | yes | yes |
| STEAP4 | yes | yes | — |
| STMN3 | yes | — | — |
| SYTL1 | yes | yes | yes |
| TBCCD1 | yes | — | — |
| TBP | yes | yes | — |
| TCEB1 | yes | — | — |
| TJP2 | yes | — | — |
| TLR2 | yes | yes | yes |
| TNFRSF10C | yes | — | — |
| TNFSF10 | yes | yes | — |
| TNFSF13B | yes | yes | — |
| TP53I13 | yes | — | — |
| TPM4 | yes | — | — |
| TPX2 | yes | yes | yes |
| TREM1 | yes | yes | — |
| TTK | yes | yes | yes |
| TXNDC5 | yes | — | — |
| TYMP | yes | yes | — |
| TYMS | yes | yes | yes |
| UBE2J1 | yes | — | — |
| VASP | yes | — | — |
| VMP1 | yes | — | — |
| WARS | yes | yes | — |
| WDR85 | yes | — | — |
| ZFP161 | yes | yes | — |
| ZNF276 | yes | yes | yes |
| ZNF384 | yes | yes | yes |
| ZNF549 | yes | — | — |

TABLE 1-5
Lyme Disease Classifier Genes

| Gene | Gene name | Weight |
|---|---|---|
| ANPEP | Alanyl Aminopeptidase, Membrane | 1.13E+00 |
| ANXA5 | Annexin A5 | −1.25E−03 |
| ASPM | Assembly Factor For Spindle Microtubules | 6.95E−03 |
| CASC5 | Cancer Susceptibility Candidate Gene 5 Protein | −1.49E−02 |
| CAV1 | Caveolin 1 | 2.74E−04 |
| CDCA5 | Cell Division Cycle-Associated Protein 5 | −1.50E−02 |
| CXCL10 | C-X-C Motif Chemokine Ligand 10 | −2.81E−03 |
| DRAM1 | DNA Damage Regulated Autophagy | −2.74E−04 |
| | Modulator 1 | |
| GBP4 | Guanylate Binding Protein 4 | 4.90E−04 |
| GRN | Granulin Precursor | −5.07E−03 |
| IFI27 | Interferon Alpha Inducible Protein 27 | −2.54E−05 |
| IFRD1 | Interferon-related developmental regulator 1 | −1.69E−03 |
| IGSF6 | Immunoglobulin superfamily member 6 | −1.22E−02 |
| ITGAM | Integrin Subunit Alpha M | 9.13E−05 |
| JMJD6 | Jumonji Domain Containing 6, Arginine | 4.22E−03 |
| | Demethylase And Lysine Hydroxylase | |
| KIF2C | Kinesin Family Member 2C | −1.87E−02 |
| LDLR | Low Density Lipoprotein Receptor | 6.35E−03 |
| MXD1 | MAX Dimerization Protein 1 | −2.13E−02 |
| NIF3L1 | NGG1 Interacting Factor 3 Like 1 | −1.51E−03 |
| PLK1 | Polo Like Kinase 1 | −1.74E−04 |
| SHCBP1 | SHC Binding And Spindle Associated 1 | 6.44E−04 |
| SOCS3 | Suppressor Of Cytokine Signaling 3 | 1.75E−04 |
| SORT1 | Sortilin 1 | −2.61E−03 |
| SPAG5 | Sperm Associated Antigen 5) | −5.30E−03 |
| STAT1 | Signal Transducer And Activator Of | −3.76E−02 |
| | Transcription 1 | |
| SYTL1 | Synaptotagmin Like 1 | 3.33E−03 |
| TLR2 | Toll Like Receptor 2 | 1.30E−03 |
| TPX2 | TPX2 Microtubule Nucleation Factor | 2.99E−03 |
| TTK | TTK Protein Kinase | 2.16E−03 |
| TYMS | Thymidylate Synthetase | −1.10E−03 |
| ZNF384 | Zinc Finger Protein 384 | −6.03E−03 |

For the independent validation test set of 63 samples, the Lyme disease gene expression classifier had an overall accuracy of 95.2% (95% [86.7%-99.0%]), with a sensitivity of 90% (95% [83.3%-100%]) and specificity of 100% (95% [90.9%-100%]) relative to two-tier Lyme antibody testing and based on misclassification of 1 Lyme seropositive and 2 Lyme seronegative samples (FIG. 2C-D). Lyme disease gene expression classifier results between subjects seropositive at presentation had higher sensitivity than those who were seropositive after 3 weeks (100% versus 83%, respectively). Lyme disease gene expression classifier sensitivities for Lyme seropositive and seronegative samples were 93.7% and 85.7%, respectively.

The 31 identified genes on the panel were related to immune cell signaling (n=7), cell division (n=6), apoptosis (n=3), cell growth and differentiation (n=3), cell trafficking (n=2), Borrelia burgdorferi receptor-binding (n=2), and 8 other functions (n=8). Many genes (23 of 31, 74.2%) had previously been described in association with cell culture (n=20), murine (n=2), and Lyme disease patient studies (n=3) of Borrelia burgdorferi infection.

Representative gene expression values shown as read counts from targeted RNA expression resequencing are provided in Table 1-6. Representative weighted gene expression values are provided in Table 1-7A and Table 1-7B.

TABLE 1-6
Representative Gene Expression Values\hat{ }

| | Subject | | | | | | | |
|---|---|---|---|---|---|---|---|---|
| | Lyme | Lyme | Healthy | Healthy | Healthy | | | |
| Gene | 1 | 2 | 1 | 2 | 3 | Bac | Flu | TB |
| ANPEP | 69.97 | 72.53 | 30.5 | 14.6 | 18.21 | 14.79 | 40.33 | 43.59 |
| ANXA5 | 438.50 | 346.27 | 79.59 | 156.82 | 152.16 | 114.42 | 12.36 | 78.51 |
| ASPM | 4.40 | 4.26 | 23.35 | 0.52 | 6.25 | 10.17 | 11.87 | 29.12 |
| CASC5 | 5.67 | 6.88 | 2.38 | 18.43 | 4.08 | 11.71 | 1.76 | 5.3 |
| CAV1 | 3.56 | 1.89 | 0 | 0.17 | 0.27 | 1.54 | 1.51 | 0.5 |
| CDCA5 | 4.27 | 0.56 | 2.86 | 0.35 | 0 | 5.78 | 1.31 | 2.69 |
| CXCL10 | 3.30 | 5.18 | 14.77 | 12.17 | 22.82 | 1.23 | 15.31 | 300.17 |
| DRAM1 | 16.27 | 24.68 | 36.7 | 11.65 | 45.38 | 15.17 | 8.19 | 32.06 |
| GBP4 | 192.07 | 274.40 | 197.3 | 270.53 | 165.48 | 151.43 | 81.31 | 214.34 |
| GRN | 240.93 | 165.75 | 337.89 | 859.75 | 170.09 | 640.28 | 209.57 | 205.84 |
| IFI27 | 0.48 | 0.30 | 0.48 | 7.65 | 11.68 | 145.04 | 116.07 | 89.88 |
| IFRD1 | 34.92 | 62.58 | 122 | 105.36 | 85.59 | 31.23 | 22.39 | 31.81 |
| IGSF6 | 467.97 | 451.55 | 312.63 | 495.85 | 336.11 | 81.49 | 157.78 | 326.51 |
| ITGAM | 136.74 | 109.61 | 82.45 | 116.49 | 31.25 | 126.48 | 73.69 | 64.46 |
| JMJD6 | 13.85 | 13.51 | 48.61 | 1.74 | 37.77 | 27.46 | 16.58 | 14.64 |
| KIF2C | 1.54 | 0.93 | 0 | 0.87 | 0 | 1.62 | 0.25 | 4.97 |
| LDLR | 21.73 | 18.17 | 3.34 | 25.38 | 24.18 | 35.59 | 6.51 | 19.19 |
| MXD1 | 0.00 | 5.03 | 81.02 | 0 | 48.64 | 0.89 | 1.35 | 35.34 |
| NIF3L1 | 1.80 | 2.52 | 28.12 | 0 | 17.39 | 0.04 | 0 | 6.14 |
| PLK1 | 14.78 | 4.22 | 33.36 | 0.35 | 17.39 | 11.86 | 12.57 | 61.6 |
| SHCBP1 | 6.82 | 5.03 | 12.87 | 0.35 | 9.24 | 14.1 | 3.52 | 17.08 |
| SOCS3 | 25.91 | 64.32 | 89.6 | 27.47 | 116.29 | 8.4 | 4.22 | 45.86 |
| SORT1 | 145.98 | 150.02 | 77.68 | 104.84 | 98.09 | 55.23 | 47.37 | 101.91 |
| SPAG5 | 4.49 | 7.25 | 21.45 | 20.52 | 15.22 | 8.59 | 0.9 | 12.62 |
| STAT1 | 262.79 | 376.50 | 630.03 | 245.32 | 467.9 | 138.72 | 907.52 | 824.53 |
| SYTL1 | 4.57 | 6.18 | 2.86 | 16.34 | 4.08 | 9.24 | 3.81 | 1.68 |
| TLR2 | 77.19 | 96.33 | 63.38 | 92.67 | 59.23 | 11.4 | 85.03 | 99.38 |
| TPX2 | 1.58 | 0.93 | 4.29 | 0.17 | 1.63 | 3.12 | 1.56 | 6.06 |
| TTK | 3.47 | 1.30 | 7.63 | 0.35 | 1.9 | 4.16 | 0.53 | 22.22 |
| TYMS | 9.54 | 4.48 | 9.53 | 0.35 | 5.16 | 11.44 | 3.89 | 29.87 |
| ZNF384 | 103.49 | 89.30 | 132.01 | 136.13 | 54.34 | 136.88 | 20.88 | 20.2 |

\hat{ }Abbreviations: Bac (bacteremia); Flu (influenza); and TB (tuberculosis).

TABLE 1-7A
Weighted Gene Expression Values for Lyme Disease and Healthy Subjects*

| | Subject | | | | | |
|---|---|---|---|---|---|---|
| | | Lyme | Lyme | Healthy | Healthy | Healthy |
| Gene | Weight | 1 | 2 | 1 | 2 | 3 |
| ANPEP | 1.1300000 | 79.06610 | 81.95890 | 34.46500 | 16.49800 | 20.57730 |
| ANXA5 | −0.0012500 | −0.54813 | −0.43284 | −0.09949 | −0.19603 | −0.19020 |
| ASPM | 0.0069500 | 0.03058 | 0.02961 | 0.16228 | 0.00361 | 0.04344 |
| CASC5 | −0.0149000 | −0.08448 | −0.10251 | −0.03546 | −0.27461 | −0.06079 |
| CAV1 | 0.0002740 | 0.00098 | 0.00052 | 0.00000 | 0.00005 | 0.00007 |
| CDCA5 | −0.0150000 | −0.06405 | −0.00840 | −0.04290 | −0.00525 | 0.00000 |
| CXCL10 | −0.0028100 | −0.00927 | −0.01456 | −0.04150 | −0.03420 | −0.06412 |
| DRAM1 | −0.0002740 | −0.00446 | −0.00676 | −0.01006 | −0.00319 | −0.01243 |
| GBP4 | 0.0004900 | 0.09411 | 0.13446 | 0.09668 | 0.13256 | 0.08109 |
| GRN | −0.0050700 | −1.22152 | −0.84035 | −1.71310 | −4.35893 | −0.86236 |
| IF127 | −0.0000254 | −0.00001 | −0.00001 | −0.00001 | −0.00019 | −0.00030 |
| IFRD1 | −0.0016900 | −0.05901 | −0.10576 | −0.20618 | −0.17806 | −0.14465 |
| IGSF6 | −0.0122000 | −5.70923 | −5.50891 | −3.81409 | −6.04937 | −4.10054 |
| ITGAM | 0.0000913 | 0.01248 | 0.01001 | 0.00753 | 0.01064 | 0.00285 |
| JMJD6 | 0.0042200 | 0.05845 | 0.05701 | 0.20513 | 0.00734 | 0.15939 |
| KIF2C | −0.0187000 | −0.02880 | −0.01739 | 0.00000 | −0.01627 | 0.00000 |
| LDLR | 0.0063500 | 0.13799 | 0.11538 | 0.02121 | 0.16116 | 0.15354 |
| MXD1 | −0.0213000 | 0.00000 | −0.10714 | −1.72573 | 0.00000 | −1.03603 |
| NIF3L1 | −0.0015100 | −0.00272 | −0.00381 | −0.04246 | 0.00000 | −0.02626 |
| PLK1 | −0.0001740 | −0.00257 | −0.00073 | −0.00580 | −0.00006 | −0.00303 |
| SHCBP1 | 0.0006440 | 0.00439 | 0.00324 | 0.00829 | 0.00023 | 0.00595 |
| SOCS3 | 0.0001750 | 0.00453 | 0.01126 | 0.01568 | 0.00481 | 0.02035 |
| SORT1 | −0.0026100 | −0.38101 | −0.39155 | −0.20274 | −0.27363 | −0.25601 |
| SPAG5 | −0.0053000 | −0.02380 | −0.03843 | −0.11369 | −0.10876 | −0.08067 |
| STAT1 | −0.0376000 | −9.88090 | −14.15640 | −23.68913 | −9.22403 | −17.59304 |
| SYTL1 | 0.0033300 | 0.01522 | 0.02058 | 0.00952 | 0.05441 | 0.01359 |
| TLR2 | 0.0013000 | 0.10035 | 0.12523 | 0.08239 | 0.12047 | 0.07700 |
| TPX2 | 0.0029900 | 0.00472 | 0.00278 | 0.01283 | 0.00051 | 0.00487 |
| TTK | 0.0021600 | 0.00750 | 0.00281 | 0.01648 | 0.00076 | 0.00410 |
| TYMS | −0.0011000 | −0.01049 | −0.00493 | −0.01048 | −0.00039 | −0.00568 |
| ZNF384 | −0.0060300 | −0.62404 | −0.53848 | −0.79602 | −0.82086 | −0.32767 |

RAW LYME DISEASE SCORE

| | | | | |
|---|---|---|---|---|
| 60.88290 | 60.19282 | 2.55418 | −4.54928 | −3.62023 |

SCALED LYME DISEASE SCORE

| | | | | |
|---|---|---|---|---|
| 0.91719 | 0.85921 | 0.02326 | 0.04495 | 0.02913 |

*Rounded to the nearest 1 × 10⁻⁷ for readability.

TABLE 1-7B
Weighted Gene Expression Values for Lyme Disease and Control Subjects*

| | Subject | | | | | |
|---|---|---|---|---|---|---|
| Gene | Weight | Lyme 1 | Lyme 2 | Bac | Flu | TB |
| ANPEP | 1.1300000 | 79.06610 | 81.95890 | 16.71270 | 45.57290 | 49.25670 |
| ANXA5 | −0.0012500 | −0.54813 | −0.43284 | −0.14303 | −0.01545 | −0.09814 |
| ASPM | 0.0069500 | 0.03058 | 0.02961 | 0.07068 | 0.08250 | 0.20238 |
| CASC5 | −0.0149000 | −0.08448 | −0.10251 | −0.17448 | −0.02622 | −0.07897 |
| CAV1 | 0.0002740 | 0.00098 | 0.00052 | 0.00042 | 0.00041 | 0.00014 |
| CDCA5 | −0.0150000 | −0.06405 | −0.00840 | −0.08670 | −0.01965 | −0.04035 |
| CXCL10 | −0.0028100 | −0.00927 | −0.01456 | −0.00346 | −0.04302 | −0.84348 |
| DRAM1 | −0.0002740 | −0.00446 | −0.00676 | −0.00416 | −0.00224 | −0.00878 |
| GBP4 | 0.0004900 | 0.09411 | 0.13446 | 0.07420 | 0.03984 | 0.10503 |
| GRN | −0.0050700 | −1.22152 | −0.84035 | −3.24622 | −1.06252 | −1.04361 |
| IFI27 | −0.0000254 | −0.00001 | −0.00001 | −0.00368 | −0.00295 | −0.00228 |
| IFRD1 | −0.0016900 | −0.05901 | −0.10576 | −0.05278 | −0.03784 | −0.05376 |
| IGSF6 | −0.0122000 | −5.70923 | −5.50891 | −0.99418 | −1.92492 | −3.98342 |
| ITGAM | 0.0000913 | 0.01248 | 0.01001 | 0.01155 | 0.00673 | 0.00589 |
| JMJD6 | 0.0042200 | 0.05845 | 0.05701 | 0.11588 | 0.06997 | 0.06178 |
| KIF2C | −0.0187000 | −0.02880 | −0.01739 | −0.03029 | −0.00468 | −0.09294 |
| LDLR | 0.0063500 | 0.13799 | 0.11538 | 0.22600 | 0.04134 | 0.12186 |
| MXD1 | −0.0213000 | 0.00000 | −0.10714 | −0.01896 | −0.02876 | −0.75274 |
| NIF3L1 | −0.0015100 | −0.00272 | −0.00381 | −0.00006 | 0.00000 | −0.00927 |
| PLK1 | −0.0001740 | −0.00257 | −0.00073 | −0.00206 | −0.00219 | −0.01072 |
| SHCBP1 | 0.0006440 | 0.00439 | 0.00324 | 0.00908 | 0.00227 | 0.01100 |
| SOCS3 | 0.0001750 | 0.00453 | 0.01126 | 0.00147 | 0.00074 | 0.00803 |
| SORT1 | −0.0026100 | −0.38101 | −0.39155 | −0.14415 | −0.12364 | −0.26599 |
| SPAG5 | −0.0053000 | −0.02380 | −0.03843 | −0.04553 | −0.00477 | −0.06689 |
| STAT1 | −0.0376000 | −9.88090 | −14.15640 | −5.21587 | −34.12275 | −31.00233 |
| SYTL1 | 0.0033300 | 0.01522 | 0.02058 | 0.03077 | 0.01269 | 0.00559 |
| TLR2 | 0.0013000 | 0.10035 | 0.12523 | 0.01482 | 0.11054 | 0.12919 |
| TPX2 | 0.0029900 | 0.00472 | 0.00278 | 0.00933 | 0.00466 | 0.01812 |
| TTK | 0.0021600 | 0.00750 | 0.00281 | 0.00899 | 0.00114 | 0.04800 |
| TYMS | −0.0011000 | −0.01049 | −0.00493 | −0.01258 | −0.00428 | −0.03286 |
| ZNF384 | −0.0060300 | −0.62404 | −0.53848 | −0.82539 | −0.12591 | −0.12181 |

RAW LYME DISEASE SCORE

| | | | | |
|---|---|---|---|---|
| 60.88290 | 60.19282 | 6.28231 | 8.39395 | 11.46537 |

SCALED LYME DISEASE SCORE

| | | | | |
|---|---|---|---|---|
| 0.91719 | 0.85921 | 0.17592 | 0.09401 | 0.06883 |

*Abbreviations: Bac (bacteremia); Flu (influenza); and TB (tuberculosis).
Rounded to the nearest 1 × 10⁻⁷ for readability

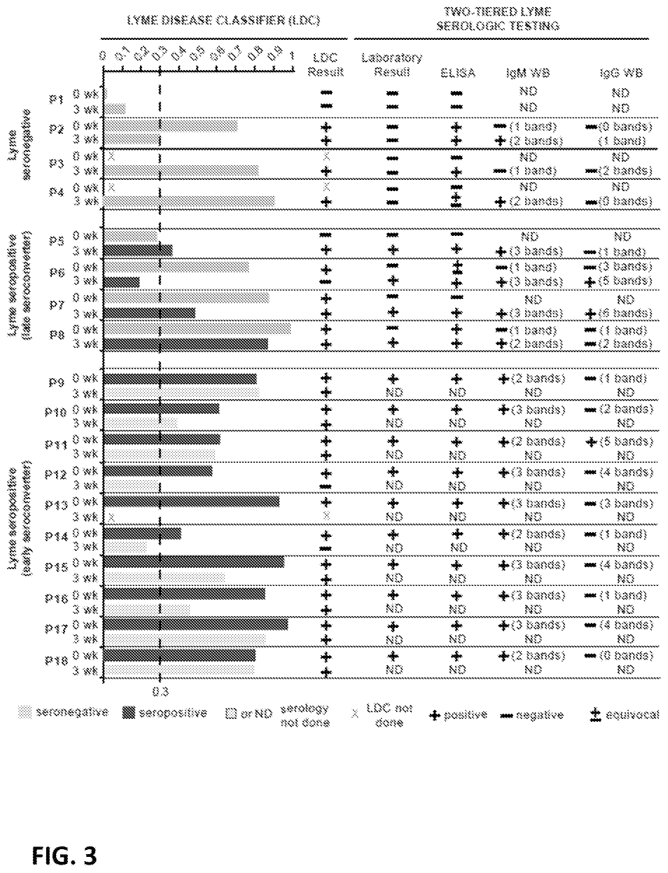

To evaluate for persistence of the Lyme disease gene expression classifier gene signature, we analyzed available serially collected samples from a subset of 18 clinical Lyme patients at 0 weeks (time of initial clinical presentation with EM rash) and 3 weeks (following completion of a 3-week course of doxycycline treatment) (FIG. 3). Among 4 Lyme seronegative cases, 3 (75%) had a discordant result, with negative Lyme serology but a positive Lyme disease classifier score of greater than or equal to 0.3 (FIG. 3, Patients 2-4). Two of these 3 cases seroconverted at 3 weeks by IgM testing (FIG. 3, Patients 2-4) but did not formally fulfill CDC criteria since the duration of illness from onset of symptoms was greater than 30 days (although would be considered seropositive using a 6-week cutoff as suggested by others) (Branda et al., Clin Infect Dis, 50:20-26, 2010), while the remaining seronegative/Lyme disease classifier-positive patient (FIG. 3, Patient 3) was ELISA positive and had one and two bands for IgM and IgG, respectively, at 3 weeks, appeared close to seroconverting, Among the 4 cases with late seroconversion 3 weeks after presentation (FIG. 3, Patients 5-8), 3 of 4 (FIG. 3, Patients 6-8) were positive by Lyme disease classifier testing at time 0 weeks, while Patient 5 was negative at 0 weeks but positive at 3 weeks. Ten of 13 cases (76.9%) that were Lyme disease classifier positive at time 0 remained persistently positive at 3 weeks (FIG. 3, Patients 2, 7-11, and 15-18), while the remaining 3 (FIG. 3, Patients 6, 12, and 14) showed a decline in the Lyme disease classifier score below the 0.3 threshold.

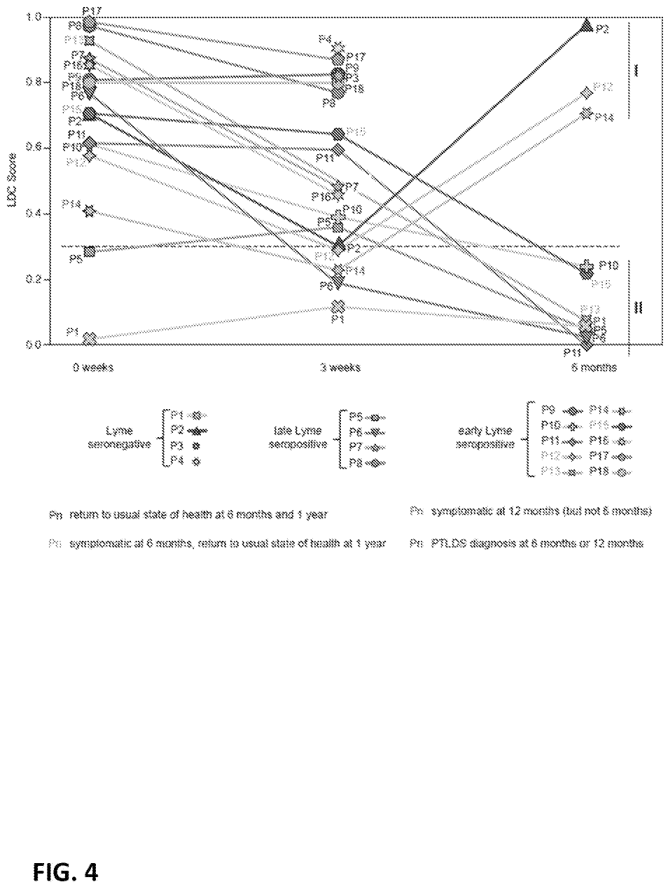

Samples from 10 patients collected at 3 weeks and/or 6 months after clinical presentation of Lyme disease were available and, based on Lyme disease classifier testing, could be assigned into two subgroups with different longitudinal trajectories (FIG. 4). One subgroup (FIG. 4, I) contained 3 patients with positive Lyme disease classifier scores at 0 weeks (FIG. 4, Patients 2, 12, and 14) that declined at 3 weeks but rebounded by 6 months. Patients 12 and 14 had persistent symptoms at 6 and 12 months, respectively, but without the functional disability to meet clinical criteria for post-treatment Lyme disease syndrome (PTLDS) (Aucott et al., Qual Life Res, 1:75-84, 2013; and Rebman and Aucott, Front Med, 7:57, 2020). The other subgroup (FIG. 4, II) contained 7 patients who had gradual declines in Lyme disease classifier scores from 0 weeks to 6 months. Among these 7 patients, 2 were symptomatic at 6 months but returned to usual state of health at 1 year (FIG. 4, Patients 13 and 16), while 1 Lyme seronegative patient diagnosed with clinical PTLDS was negative by Lyme disease classifier testing at all 3 time points (FIG. 4, Patient 1). Unfortunately, 6-month samples were not available for two Lyme disease patients who met clinical criteria for PTLDS and had a persistently positive Lyme disease classifier signature at 3 weeks (FIG. 4, Patients 4 and 9).

Various modifications and variations of the present disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure which are understood by those skilled in the art are intended to be within the scope of the claims.

We claim:

1. A method for measuring gene expression, comprising the steps of:
(a) measuring RNA expression of a plurality of genes of cells from a blood sample obtained from a mammalian subject suspected of having a tick-borne disease;
(b) calculating a weighted RNA expression score for each of the plurality of genes; and
(c) calculating a Lyme disease score by taking the sum of the weighted RNA expression scores, wherein the plurality of genes comprises at least 3, 4, 5, 6 or all 7 genes of the group consisting of IGSF6, JMJD6, NIF3L1, SHCBP1, SYTL1, TTK and ZNF384.

2. The method of claim 1, wherein the plurality of genes further comprises at least 1, 2, 3, 4, 5, 6, 7, 8, 9, 10, 11, 12, 13, 14, 15, 16, 17, 18, 19 or all 20 genes of the group consisting of ANPEP, ASPM, CASC5, CAV1, CDCA5, CXCL10, DRAM1, GBP4, GRN, IFRD1, KIF2C, LDLR, MXD1, SOCS3, SORT1, SPAG5, STAT1, TLR2, TPX2 and TYMS.

3. The method of claim 2, wherein the plurality of genes further comprises at least 1, 2, 3 or all 4 of the group consisting of ANXA5, IFI27, ITGAM and PLK1.

4. The method of claim 1, wherein the plurality of genes comprises all 31 genes of the group consisting of ANPEP, ANXA5, ASPM, CASC5, CAV1, CDCA5, CXCL10, DRAM1, GBP4, GRN, IFI27, IFRD1, IGSF6, ITGAM, JMJD6, KIF2C, LDLR, MXD1, NIF3L1, PLK1, SHCBP1, SOCS3, SORT1, SPAG5, STAT1, SYTL1, TLR2, TPX2, TTK, TYMS and ZNF384.

5. The method of claim 4, for providing information to assess whether a subject has early Lyme disease, further comprising: step (d) identifying the subject as not having early Lyme disease when the Lyme disease score is below a threshold value; or identifying the subject as having early Lyme disease when the Lyme disease score is above a threshold value.

6. The method of claim 5, wherein the threshold value is a maximum Youden's index value as plotted on an area under curve-receiver operating characteristic curve (AUC-ROC) that maximizes the accuracy of the LDC, optionally wherein the AUC-ROC is determined by use of a generalized linear model.

7. The method of claim 6, further comprising: obtaining a blood sample from the subject prior to step (a).

8. The method of claim 7, wherein the cells are peripheral blood mononuclear cells (PBMCs) isolated from the blood sample or wherein the cells are whole blood cells, optionally wherein the cells are peripheral blood mononuclear cells (PBMCs) isolated from the blood sample.

9. The method of claim 8, further comprising: extracting RNA from the cells prior to step (a).

10. The method of claim 9, wherein step (a) comprises one or more of the group consisting of sequence analysis, hybridization, and amplification.

11. The method of claim 10, wherein step (a) comprises targeted RNA expression resequencing comprising:
(i) preparing an RNA expression library for the plurality of targeted genes from RNA extracted from the cells;
(ii) sequencing a portion of at least 50,000 members of the library; and
(iii) generating a read count for RNA expression of the plurality of genes by normalization to the sequence of the at least 50,000 members of step (ii).

12. The method of claim 10, wherein step (a) comprises whole transcriptome shotgun sequencing (WTSS) comprising:
(i) preparing an RNA expression library for the plurality of genes from RNA extracted from the cells;
(ii) sequencing a portion of at least 1,000,000 members of the library; and
(iii) generating a read count for RNA expression of the plurality of genes by normalization to the sequence of the at least 1,000,000 members of step (ii).

13. The method of claim 11 or claim 12, wherein step (b) comprises: multiplying the read count for each of the plurality of genes by a predetermined gene expression weight to obtain the weighted RNA expression score.

14. The method of claim 10, wherein step (a) comprises: performing a nucleic acid amplification technique (NAAT) on RNA extracted from the PBMCs, wherein the NAAT comprises a thermal cycle amplification technique or an isothermal amplification technique.

15. The method of claim 14, wherein step (a) comprises performing a thermal cycle amplification technique on RNA extracted from the PBMCs, wherein the thermal cycle amplification technique is selected from the group consisting of reverse transcriptase-quantitative polymerase chain reaction (RT-qPCR) and reverse transcriptase-digital polymerase chain reaction (RT-dPCR).

16. The method of claim 14, wherein step (a) comprises performing an isothermal amplification technique on RNA extracted from the PBMCs, wherein the isothermal amplification technique is selected from the group consisting of reverse transcriptase-loop-mediated isothermal amplification (RT-LAMP) and reverse transcriptase-recombinase polymerase amplification (RT-RPA).

17. The method of claim 10, wherein step (a) comprises: hybridizing RNA extracted from the cells to a microarray.

18. The method of claim 10, wherein step (a) comprises: performing serial amplification of gene expression (SAGE) on RNA extracted from the cells.

19. The method of claim 1, wherein the subject was bitten by a tick in a region where at least 20% of ticks are suspected of being infected with Borrelia burgdorferi.

20. The method of claim 19, wherein the subject was bitten by a tick within three weeks of the blood sample being obtained.

21. The method of claim 20, wherein the subject has an erythema migrans rash when the blood sample was obtained.

22. The method of claim 20, wherein the subject does not have an erythema migrans rash when the blood sample was obtained.

23. The method of claim 21 or claim 22, wherein the subject has flu-like symptoms when the blood sample was obtained.

24. The method of claim 1, further comprising performing a serologic test for Lyme disease.

25. The method of claim 24, wherein the subject was determined to be negative for Lyme disease by serologic testing at the time the blood sample was obtained.

26. The method of claim 1, wherein the tick-borne disease is selected from the group consisting of Borreliosis, Southern tick associated rash illness, Q fever, Colorado tick fever, Powassan virus infection, tick-borne encephalitis virus infection, tick-borne relapsing fever, Heartland virus infection and severe fever with thrombocytopenia virus infection.

27. The method of claim 5, further comprising:
step (e) administering an antibiotic therapy to the subject to treat the Lyme disease when the subject has been identified as having early Lyme disease.

28. The method of claim 27, wherein the antibiotic therapy comprises:
(i) an effective amount of an antibiotic selected from the group consisting of tetracyclines, penicillins, and cephalosporins or
(ii) an oral regimen comprising doxycycline, amoxicillin or cefuroxime axetil; or
(iii) a parenteral regimen comprising ceftriaxone, cefotaxime, or penicillin G.

29. An antibiotic therapy for use in a method of treating Lyme disease in a subject that has been identified as having acute Lyme disease according to the method of claim 5.

30. The antibiotic therapy for the use of claim 28, wherein the antibiotic therapy comprises:
(i) an effective amount of an antibiotic selected from the group consisting of tetracyclines, penicillins, and cephalosporins or
(ii) an oral regimen comprising doxycycline, amoxicillin or cefuroxime axetil; or
(iii) a parenteral regimen comprising ceftriaxone, cefotaxime, or penicillin G.

31. A kit comprising:
(a) a plurality of oligonucleotides which hybridize to a plurality of genes comprising at least 3, 4, 5, 6, or 7 genes of the group consisting of IGSF6, JMJD6, NIF3L1, SHCBP1, SYTL1, TTK and ZNF384;
(b) instructions and/or an algorithm for: (i) use of the oligonucleotides for measuring RNA expression of the plurality of genes; (ii) calculating a weighted RNA expression score for each of the plurality of genes; and (iii) calculating a Lyme disease score by taking the sum of the weighted RNA expression scores.

32. The kit of claim 31, wherein the plurality of genes further comprises at least 1, 2, 3, 4, 5, 6, 7, 8, 9, 10, 11, 12, 13, 14, 15, 16, 17, 18, 19 or all 20 genes of the group consisting of ANPEP, ASPM, CASC5, CAV1, CDCA5, CXCL10, DRAM1, GBP4, GRN4, IFRD1, KIF2C, LDLR, MXD1, SOCS3, SORT1, SPAG5, STAT1, TLR2, TPX2 and TYMS.

33. The kit of claim 32, wherein the plurality of genes further comprises at least 1, 2, 3 or all 4 of the group consisting of ANXA5, IFI27, ITGAM and PLK1.

34. The kit of claim 31, wherein the plurality of genes comprises all 31 genes of the group consisting of ANPEP, ANXA5, ASPM, CASC5, CAV1, CDCA5, CXCL10, DRAM1, GBP4, GRN, IFI27, IFRD1, IGSF6, ITGAM, JMJD6, KIF2C, LDLR, MXD1, NIF3L1, PLK1, SHCBP1, SOCS3, SORT1, SPAG5, STAT1, SYTL1, TLR2, TPX2, TTK, TYMS and ZNF384.

35. An in vitro method for measuring RNA expression of a gene, comprising:
i) obtaining RNA extracted from a peripheral blood mononuclear cell (PBMC) sample of a human subject suspected of having a tick-borne disease; and
ii) performing targeted RNA expression resequencing comprising hybridization of an upstream oligonucleotide and a downstream oligonucleotide to the extracted RNA to measure RNA expression of the gene,
wherein the gene comprises one or more of the group consisting of IGSF6, JMJD6, NIF3L1, SHCBP1, SYTL1, TTK and ZNF384.

36. The method of claim 35, wherein the gene comprises IGSF6 and the upstream oligonucleotide comprises the nucleotide sequence of SEQ ID NO:25 and the downstream oligonucleotide comprises the nucleotide sequence of SEQ ID NO:26.

37. The method of claim 35, wherein the gene comprises JMJD6 and the upstream oligonucleotide comprises the nucleotide sequence of SEQ ID NO:29 and the downstream oligonucleotide comprises the nucleotide sequence of SEQ ID NO:30.

38. The method of claim 35, wherein the gene comprises NIF3L1 and the upstream oligonucleotide comprises the nucleotide sequence of SEQ ID NO:37 and the downstream oligonucleotide comprises the nucleotide sequence of SEQ ID NO:38.

39. The method of claim 35, wherein the gene comprises SHCBP1 and the upstream oligonucleotide comprises the nucleotide sequence of SEQ ID NO:41 and the downstream oligonucleotide comprises the nucleotide sequence of SEQ ID NO:42.

40. The method of claim 35, wherein the gene comprises SYTL1 and the upstream oligonucleotide comprises the nucleotide sequence of SEQ ID NO:51 and the downstream oligonucleotide comprises the nucleotide sequence of SEQ ID NO:52.

41. The method of claim 35, wherein the gene comprises a plurality of genes comprises at least 3, 4, 5, 6 or all 7 of the group consisting of IGSF6, JMJD6, NIF3L1, SHCBP1, SYTL1, TTK and ZNF384, the upstream oligonucleotide comprises a plurality of upstream oligonucleotides, and the downstream oligonucleotide comprise a plurality of oligonucleotides, and
wherein when the plurality of genes comprises:
IGSF6, one of the plurality of upstream oligonucleotides comprises the nucleotide sequence of SEQ ID NO:25 and one of the plurality of downstream oligonucleotides comprises the nucleotide sequence of SEQ ID NO:26;
JMJD6, one of the plurality of upstream oligonucleotides comprises the nucleotide sequence of SEQ ID NO:29 and one of the plurality of downstream oligonucleotides comprises the nucleotide sequence of SEQ ID NO:30;
NIF3L1, one of the plurality of upstream oligonucleotides comprises the nucleotide sequence of SEQ ID NO:37 and one of the plurality of downstream oligonucleotides comprises the nucleotide sequence of SEQ ID NO:38;
SHCBP1, one of the plurality of upstream oligonucleotides comprises the nucleotide sequence of SEQ ID NO:41 and one of the plurality of downstream oligonucleotides comprises the nucleotide sequence of SEQ ID NO:42;
SYTL1, one of the plurality of upstream oligonucleotides comprises the nucleotide sequence of SEQ ID NO:51 and one of the plurality of downstream oligonucleotides comprises the nucleotide sequence of SEQ ID NO:52;
TTK, one of the plurality of upstream oligonucleotides comprises the nucleotide sequence of SEQ ID NO:57 and one of the plurality of downstream oligonucleotides comprises the nucleotide sequence of SEQ ID NO:58; and/or
ZNF384, one of the plurality of upstream oligonucleotides comprises the nucleotide sequence of SEQ ID NO:61 and one of the plurality of downstream oligonucleotides comprises the nucleotide sequence of SEQ ID NO:62.

42. The method of claim 41, wherein the plurality of genes comprises all 7 of the group consisting of IGSF6, JMJD6, NIF3L1, SHCBP1, SYTL1, TTK and ZNF384.

43. The method of claim 42, wherein the plurality of genes further comprises at least 1, 2, 3, 4, 5, 6, 7, 8, 9, 10, 11, 12, 13, 14, 15, 16, 17, 18, 19 or all 20 of the group consisting of ANPEP, ASPM, CASC5, CAV1, CDCA5, CXCL10, DRAM1, GBP4, GRN, IFRD1, KIF2C, LDLR, MXD1, SOCS3, SORT1, SPAG5, STAT1, TLR2, TPX2 and TYMS, and
wherein when the plurality of genes comprises:
ANPEP, one of the plurality of upstream oligonucleotides comprises the nucleotide sequence of SEQ ID NO:1 and one of the plurality of downstream oligonucleotides comprises the nucleotide sequence of SEQ ID NO:2;
ASPM, one of the plurality of upstream oligonucleotides comprises the nucleotide sequence of SEQ ID NO:5 and one of the plurality of downstream oligonucleotides comprises the nucleotide sequence of SEQ ID NO:6;
CASC5, one of the plurality of upstream oligonucleotides comprises the nucleotide sequence of SEQ ID NO:7 and one of the plurality of downstream oligonucleotides comprises the nucleotide sequence of SEQ ID NO:8;
CAV1, one of the plurality of upstream oligonucleotides comprises the nucleotide sequence of SEQ ID NO:9 and one of the plurality of downstream oligonucleotides comprises the nucleotide sequence of SEQ ID NO:10;
CDCA5, one of the plurality of upstream oligonucleotides comprises the nucleotide sequence of SEQ ID NO: 11 and one of the plurality of downstream oligonucleotides comprises the nucleotide sequence of SEQ ID NO: 12;
CXCL10, one of the plurality of upstream oligonucleotides comprises the nucleotide sequence of SEQ ID NO: 13 and one of the plurality of downstream oligonucleotides comprises the nucleotide sequence of SEQ ID NO:14;
DRAM1, one of the plurality of upstream oligonucleotides comprises the nucleotide sequence of SEQ ID NO: 15 and one of the plurality of downstream oligonucleotides comprises the nucleotide sequence of SEQ ID NO:16;
GBP4, one of the plurality of upstream oligonucleotides comprises the nucleotide sequence of SEQ ID NO:17 and one of the plurality of downstream oligonucleotides comprises the nucleotide sequence of SEQ ID NO: 18;
GRN, one of the plurality of upstream oligonucleotides comprises the nucleotide sequence of SEQ ID NO:19 and one of the plurality of downstream oligonucleotides comprises the nucleotide sequence of SEQ ID NO:20;
IFRD1, one of the plurality of upstream oligonucleotides comprises the nucleotide sequence of SEQ ID NO:23 and one of the plurality of downstream oligonucleotides comprises the nucleotide sequence of SEQ ID NO:24;
KIF2C, one of the plurality of upstream oligonucleotides comprises the nucleotide sequence of SEQ ID NO:31 and one of the plurality of downstream oligonucleotides comprises the nucleotide sequence of SEQ ID NO:32;
LDLR, one of the plurality of upstream oligonucleotides comprises the nucleotide sequence of SEQ ID NO:33 and one of the plurality of downstream oligonucleotides comprises the nucleotide sequence of SEQ ID NO:34;
MXD1, one of the plurality of upstream oligonucleotides comprises the nucleotide sequence of SEQ ID NO:35 and one of the plurality of downstream oligonucleotides comprises the nucleotide sequence of SEQ ID NO:36;
SOCS3, one of the plurality of upstream oligonucleotides comprises the nucleotide sequence of SEQ ID NO:43 and one of the plurality of downstream oligonucleotides comprises the nucleotide sequence of SEQ ID NO:44;
SORT1, one of the plurality of upstream oligonucleotides comprises the nucleotide sequence of SEQ ID NO:45 and one of the plurality of downstream oligonucleotides comprises the nucleotide sequence of SEQ ID NO:46;
SPAG5, one of the plurality of upstream oligonucleotides comprises the nucleotide sequence of SEQ ID NO:47 and one of the plurality of downstream oligonucleotides comprises the nucleotide sequence of SEQ ID NO:48;
STAT1, one of the plurality of upstream oligonucleotides comprises the nucleotide sequence of SEQ ID NO:49 and one of the plurality of downstream oligonucleotides comprises the nucleotide sequence of SEQ ID NO:50;
TLR2, one of the plurality of upstream oligonucleotides comprises the nucleotide sequence of SEQ ID NO:53 and one of the plurality of downstream oligonucleotides comprises the nucleotide sequence of SEQ ID NO:54;
TPX2, one of the plurality of upstream oligonucleotides comprises the nucleotide sequence of SEQ ID NO:55 and one of the plurality of downstream oligonucleotides comprises the nucleotide sequence of SEQ ID NO:56; and/or
TYMS, one of the plurality of upstream oligonucleotides comprises the nucleotide sequence of SEQ ID NO:59 and one of the plurality of downstream oligonucleotides comprises the nucleotide sequence of SEQ ID NO:60.

44. The method of claim 43, wherein the plurality of genes further comprises all 20 of the group consisting of ANPEP, ASPM, CASC5, CAV1, CDCA5, CXCL10, DRAM1, GBP4, GRN, IFRD1, KIF2C, LDLR, MXD1, SOCS3, SORT1, SPAG5, STAT1, TLR2, TPX2 and TYMS.

45. The method of claim 44, wherein the plurality of genes further comprises at least 1, 2, 3 or all 4 of the group consisting of ANXA5, IFI27, ITGAM and PLK1, and wherein when the plurality of genes comprises:
ANXA5, one of the plurality of upstream oligonucleotides comprises the nucleotide sequence of SEQ ID NO:3 and one of the plurality of downstream oligonucleotides comprises the nucleotide sequence of SEQ ID NO:4;
IFI27, one of the plurality of upstream oligonucleotides comprises the nucleotide sequence of SEQ ID NO:21 and one of the plurality of downstream oligonucleotides comprises the nucleotide sequence of SEQ ID NO:22;
ITGAM, one of the plurality of upstream oligonucleotides comprises the nucleotide sequence of SEQ ID NO:27 and one of the plurality of downstream oligonucleotides comprises the nucleotide sequence of SEQ ID NO:28 and/or
PLK1, one of the plurality of upstream oligonucleotides comprises the nucleotide sequence of SEQ ID NO:39 and one of the plurality of downstream oligonucleotides comprises the nucleotide sequence of SEQ ID NO:40.

46. The method of claim 45, wherein the plurality of genes further comprises all 4 of the group consisting of ANXA5, IFI27, ITGAM and PLK1.

47. A kit comprising:
(a) a plurality of upstream oligonucleotides and a plurality of downstream oligonucleotides, which hybridize to one of a plurality of genes comprising at least 3, 4, 5, 6, or 7 of the group consisting of IGSF6, JMJD6, NIF3L1, SHCBP1, SYTL1, TTK and ZNF384;
(b) instructions for: (i) use of the oligonucleotides for measuring RNA expression of the plurality of genes by performing targeted RNA expression resequencing comprising hybridization of the plurality of upstream oligonucleotides and the plurality of a downstream oligonucleotides to RNA extracted from a peripheral blood mononuclear cell (PBMC) sample of a human subject suspected of having a tick-bome disease to measure RNA expression of the plurality of genes,
wherein when the plurality of genes comprises:
IGSF6, one of the plurality of upstream oligonucleotides comprises the nucleotide sequence of SEQ ID NO:25 and one of the plurality of downstream oligonucleotides comprises the nucleotide sequence of SEQ ID NO:26;
JMJD6, one of the plurality of upstream oligonucleotides comprises the nucleotide sequence of SEQ ID NO:29 and one of the plurality of downstream oligonucleotides comprises the nucleotide sequence of SEQ ID NO:30;
NIF3L1, one of the plurality of upstream oligonucleotides comprises the nucleotide sequence of SEQ ID NO:37 and one of the plurality of downstream oligonucleotides comprises the nucleotide sequence of SEQ ID NO:38;
SHCBP1, one of the plurality of upstream oligonucleotides comprises the nucleotide sequence of SEQ ID NO:41 and one of the plurality of downstream oligonucleotides comprises the nucleotide sequence of SEQ ID NO:42;
SYTL1, one of the plurality of upstream oligonucleotides comprises the nucleotide sequence of SEQ ID NO:51 and one of the plurality of downstream oligonucleotides comprises the nucleotide sequence of SEQ ID NO:52;
TTK, one of the plurality of upstream oligonucleotides comprises the nucleotide sequence of SEQ ID NO:57 and one of the plurality of downstream oligonucleotides comprises the nucleotide sequence of SEQ ID NO:58; and/or
ZNF384, one of the plurality of upstream oligonucleotides comprises the nucleotide sequence of SEQ ID NO:61 and one of the plurality of downstream oligonucleotides comprises the nucleotide sequence of SEQ ID NO:62.

48. The kit of claim 47, wherein the plurality of genes comprises all 7 of the group consisting of IGSF6, JMJD6, NIF3L1, SHCBP1, SYTL1, TTK and ZNF384.

49. The kit of claim 48, wherein the plurality of genes further comprises at least 1, 2, 3, 4, 5, 6, 7, 8, 9, 10, 11, 12, 13, 14, 15, 16, 17, 18, 19 or all 20 of the group consisting of ANPEP, ASPM, CASC5, CAV1, CDCA5, CXCL10, DRAM1, GBP4, GRN4, IFRD1, KIF2C, LDLR, MXD1, SOCS3, SORT1, SPAG5, STAT1, TLR2, TPX2 and TYMS, and
wherein when the plurality of genes comprises:
ANPEP, one of the plurality of upstream oligonucleotides comprises the nucleotide sequence of SEQ ID NO: 1 and one of the plurality of downstream oligonucleotides comprises the nucleotide sequence of SEQ ID NO:2;
ASPM, one of the plurality of upstream oligonucleotides comprises the nucleotide sequence of SEQ ID NO:5 and one of the plurality of downstream oligonucleotides comprises the nucleotide sequence of SEQ ID NO:6;
CASC5, one of the plurality of upstream oligonucleotides comprises the nucleotide sequence of SEQ ID NO:7 and one of the plurality of downstream oligonucleotides comprises the nucleotide sequence of SEQ ID NO:8;
CAV1, one of the plurality of upstream oligonucleotides comprises the nucleotide sequence of SEQ ID NO:9 and one of the plurality of downstream oligonucleotides comprises the nucleotide sequence of SEQ ID NO: 10;
CDCA5, one of the plurality of upstream oligonucleotides comprises the nucleotide sequence of SEQ ID NO: 11 and one of the plurality of downstream oligonucleotides comprises the nucleotide sequence of SEQ ID NO:12;
CXCL10, one of the plurality of upstream oligonucleotides comprises the nucleotide sequence of SEQ ID NO:13 and one of the plurality of downstream oligonucleotides comprises the nucleotide sequence of SEQ ID NO:14;
DRAM1, one of the plurality of upstream oligonucleotides comprises the nucleotide sequence of SEQ ID NO:15 and one of the plurality of downstream oligonucleotides comprises the nucleotide sequence of SEQ ID NO:16;
GBP4, one of the plurality of upstream oligonucleotides comprises the nucleotide sequence of SEQ ID NO:17 and one of the plurality of downstream oligonucleotides comprises the nucleotide sequence of SEQ ID NO:18;
GRN, one of the plurality of upstream oligonucleotides comprises the nucleotide sequence of SEQ ID NO: 19 and one of the plurality of downstream oligonucleotides comprises the nucleotide sequence of SEQ ID NO:20;
IFRD1, one of the plurality of upstream oligonucleotides comprises the nucleotide sequence of SEQ ID NO:23 and one of the plurality of downstream oligonucleotides comprises the nucleotide sequence of SEQ ID NO:24;
KIF2C, one of the plurality of upstream oligonucleotides comprises the nucleotide sequence of SEQ ID NO:31 and one of the plurality of downstream oligonucleotides comprises the nucleotide sequence of SEQ ID NO:32;
LDLR, one of the plurality of upstream oligonucleotides comprises the nucleotide sequence of SEQ ID NO:33 and one of the plurality of downstream oligonucleotides comprises the nucleotide sequence of SEQ ID NO:34;
MXD1, one of the plurality of upstream oligonucleotides comprises the nucleotide sequence of SEQ ID NO:35 and one of the plurality of downstream oligonucleotides comprises the nucleotide sequence of SEQ ID NO:36;
SOCS3, one of the plurality of upstream oligonucleotides comprises the nucleotide sequence of SEQ ID NO:43 and one of the plurality of downstream oligonucleotides comprises the nucleotide sequence of SEQ ID NO:44;
SORT1, one of the plurality of upstream oligonucleotides comprises the nucleotide sequence of SEQ ID NO:45 and one of the plurality of downstream oligonucleotides comprises the nucleotide sequence of SEQ ID NO:46;
SPAG5, one of the plurality of upstream oligonucleotides comprises the nucleotide sequence of SEQ ID NO:47 and one of the plurality of downstream oligonucleotides comprises the nucleotide sequence of SEQ ID NO:48;
STAT1, one of the plurality of upstream oligonucleotides comprises the nucleotide sequence of SEQ ID NO:49 and one of the plurality of downstream oligonucleotides comprises the nucleotide sequence of SEQ ID NO:50;
TLR2, one of the plurality of upstream oligonucleotides comprises the nucleotide sequence of SEQ ID NO:53 and one of the plurality of downstream oligonucleotides comprises the nucleotide sequence of SEQ ID NO:54;
TPX2, one of the plurality of upstream oligonucleotides comprises the nucleotide sequence of SEQ ID NO:55 and one of the plurality of downstream oligonucleotides comprises the nucleotide sequence of SEQ ID NO:56; and/or
TYMS, one of the plurality of upstream oligonucleotides comprises the nucleotide sequence of SEQ ID NO:59 and one of the plurality of downstream oligonucleotides comprises the nucleotide sequence of SEQ ID NO:60.

50. The kit of claim 49, wherein the plurality of genes further comprises all 20 of the group consisting of ANPEP, ASPM, CASC5, CAV1, CDCA5, CXCL10, DRAM1, GBP4, GRN, IFRD1, KIF2C, LDLR, MXD1, SOCS3, SORT1, SPAG5, STAT1, TLR2, TPX2 and TYMS.

51. The kit of claim 50, wherein the plurality of genes further comprises at least 1, 2, 3 or all 4 of the group consisting of ANXA5, IFI27, ITGAM and PLK1, and
wherein when the plurality of genes comprises:
ANXA5, one of the plurality of upstream oligonucleotides comprises the nucleotide sequence of SEQ ID NO:3 and one of the plurality of downstream oligonucleotides comprises the nucleotide sequence of SEQ ID NO:4;
IFI27, one of the plurality of upstream oligonucleotides comprises the nucleotide sequence of SEQ ID NO:21 and one of the plurality of downstream oligonucleotides comprises the nucleotide sequence of SEQ ID NO:22;
ITGAM, one of the plurality of upstream oligonucleotides comprises the nucleotide sequence of SEQ ID NO:27 and one of the plurality of downstream oligonucleotides comprises the nucleotide sequence of SEQ ID NO:28 and/or PLK1, one of the plurality of upstream oligonucleotides comprises the nucleotide sequence of SEQ ID NO:39 and one of the plurality of downstream oligonucleotides comprises the nucleotide sequence of SEQ ID NO:40.

52. The kit of claim 51, wherein the plurality of genes further comprises all 4 of the group consisting of ANXA5, IFI27, ITGAM and PLK1.

53. The kit of any one of claims 47-52, further comprising:
(c) an algorithm for calculating a weighted RNA expression score for each of the plurality of genes and calculating a Lyme disease score by taking the sum of the weighted RNA expression scores.


SEQUENCE LISTING (62 sequences)

SEQ ID NO: 1 - DNA, 23 bp, synthetic construct
  source 1..23: mol_type = other DNA
ttctccaccgagtatgagctgca

SEQ ID NO: 2 - DNA, 27 bp, synthetic construct
  source 1..27: mol_type = other DNA
gagcagttcaagaaggacaacgaggaa

SEQ ID NO: 3 - DNA, 27 bp, synthetic construct
  source 1..27: mol_type = other DNA
agaattttgccacctctctttattcca

SEQ ID NO: 4 - DNA, 27 bp, synthetic construct
  source 1..27: mol_type = other DNA
gactataagaaagctcttctgctgctc

SEQ ID NO: 5 - DNA, 25 bp, synthetic construct
  source 1..25: mol_type = other DNA
gtgtgtcgcagcgctgtttcgtgtt

SEQ ID NO: 6 - DNA, 25 bp, synthetic construct
  source 1..25: mol_type = other DNA
tttctgttaactggacaccactcaa

SEQ ID NO: 7 - DNA, 25 bp, synthetic construct
  source 1..25: mol_type = other DNA
cgcaaaatttgtcgaggaggtttgc

SEQ ID NO: 8 - DNA, 27 bp, synthetic construct
  source 1..27: mol_type = other DNA
atggatggggtgtcttcagaggctaat

SEQ ID NO: 9 - DNA, 25 bp, synthetic construct
  source 1..25: mol_type = other DNA
aacacctcaacgatgacgtggtcaa

SEQ ID NO: 10 - DNA, 25 bp, synthetic construct
  source 1..25: mol_type = other DNA
actttgaagatgtgattgcagaacc

SEQ ID NO: 11 - DNA, 23 bp, synthetic construct
  source 1..23: mol_type = other DNA
cccgagcatcctccctgaaatct

SEQ ID NO: 12 - DNA, 25 bp, synthetic construct
  source 1..25: mol_type = other DNA
gtgcggctgcagtcagaaagcccat

SEQ ID NO: 13 - DNA, 28 bp, synthetic construct
  source 1..28: mol_type = other DNA
ctgccttatctttctgactctaagtggc

SEQ ID NO: 14 - DNA, 24 bp, synthetic construct
  source 1..24: mol_type = other DNA
gaactgtacgctgtacctgcatca

SEQ ID NO: 15 - DNA, 30 bp, synthetic construct
  source 1..30: mol_type = other DNA
ttctacttcctaactttcatccaagatttc

SEQ ID NO: 16 - DNA, 27 bp, synthetic construct
  source 1..27: mol_type = other DNA
atccacagaaatcaatggtgatatttg

SEQ ID NO: 17 - DNA, 27 bp, synthetic construct
  source 1..27: mol_type = other DNA
gagcatgaaaggctgctaaaacacaag

SEQ ID NO: 18 - DNA, 29 bp, synthetic construct
  source 1..29: mol_type = other DNA
aagaaatgcttaaggaagaatttcaaaag

SEQ ID NO: 19 - DNA, 24 bp, synthetic construct
  source 1..24: mol_type = other DNA
agggacttccagttgctgcccctt

SEQ ID NO: 20 - DNA, 25 bp, synthetic construct
  source 1..25: mol_type = other DNA
gtggcatgcggggatggccatcact

SEQ ID NO: 21 - DNA, 26 bp, synthetic construct
  source 1..26: mol_type = other DNA
tcagcttcacattctcaggaactctc

SEQ ID NO: 22 - DNA, 26 bp, synthetic construct
  source 1..26: mol_type = other DNA
tctggctgaagttgaggatctcttac

SEQ ID NO: 23 - DNA, 28 bp, synthetic construct
  source 1..28: mol_type = other DNA
ttgaagggattaattgacctaaccctgg

SEQ ID NO: 24 - DNA, 26 bp, synthetic construct
  source 1..26: mol_type = other DNA
acaaggcaagcagctcttgaaggtat

SEQ ID NO: 25 - DNA, 29 bp, synthetic construct
  source 1..29: mol_type = other DNA
accataagagacatgtggaaacaaatcag

SEQ ID NO: 26 - DNA, 29 bp, synthetic construct
  source 1..29: mol_type = other DNA
agaaagataacaacacttatgaaaacaga

SEQ ID NO: 27 - DNA, 28 bp, synthetic construct
  source 1..28: mol_type = other DNA
gccatggctctcagagtccttctgttaa

SEQ ID NO: 28 - DNA, 25 bp, synthetic construct
  source 1..25: mol_type = other DNA
gttcaacttggacactgaaaacgca

SEQ ID NO: 29 - DNA, 24 bp, synthetic construct
  source 1..24: mol_type = other DNA
tactacgagagcttctcgctgagc

SEQ ID NO: 30 - DNA, 25 bp, synthetic construct
  source 1..25: mol_type = other DNA
tggaaagggcagatgctttacagct

SEQ ID NO: 31 - DNA, 27 bp, synthetic construct
  source 1..27: mol_type = other DNA
tttcccggtctcgctatcaagatccaa

SEQ ID NO: 32 - DNA, 27 bp, synthetic construct
  source 1..27: mol_type = other DNA
taattcacagtgccaatgtaaggactg

SEQ ID NO: 33 - DNA, 23 bp, synthetic construct
  source 1..23: mol_type = other DNA
gtgagggctctgtccattgtcct

SEQ ID NO: 34 - DNA, 24 bp, synthetic construct
  source 1..24: mol_type = other DNA
tttgcctgggggtcttccttctat

SEQ ID NO: 35 - DNA, 22 bp, synthetic construct
  source 1..22: mol_type = other DNA
aggcggccgactatctggagcg

SEQ ID NO: 36 - DNA, 27 bp, synthetic construct
  source 1..27: mol_type = other DNA
atggttatgcctccatgttaccataca

SEQ ID NO: 37 - DNA, 28 bp, synthetic construct
  source 1..28: mol_type = other DNA
ccagggcgtcaacaactggttggctaaa

SEQ ID NO: 38 - DNA, 23 bp, synthetic construct
  source 1..23: mol_type = other DNA
ggcccatacatccttccaaagct

SEQ ID NO: 39 - DNA, 23 bp, synthetic construct
  source 1..23: mol_type = other DNA
gcagcgtgcagatcaacttcttc

SEQ ID NO: 40 - DNA, 23 bp, synthetic construct
  source 1..23: mol_type = other DNA
acaccaagctcatcttgtgccca

SEQ ID NO: 41 - DNA, 25 bp, synthetic construct
  source 1..25: mol_type = other DNA
acctgcaagaaaatgctgaagatgg

SEQ ID NO: 42 - DNA, 27 bp, synthetic construct
  source 1..27: mol_type = other DNA
taaaattcagacaagtggagagccaga

SEQ ID NO: 43 - DNA, 26 bp, synthetic construct
  source 1..26: mol_type = other DNA
gctggactgctgccaccccgggtgct

SEQ ID NO: 44 - DNA, 28 bp, synthetic construct
  source 1..28: mol_type = other DNA
gccatagcactgatcagtgacaatttac

SEQ ID NO: 45 - DNA, 29 bp, synthetic construct
  source 1..29: mol_type = other DNA
gtgctcattgtgaagaaatatgtctgtgg

SEQ ID NO: 46 - DNA, 24 bp, synthetic construct
  source 1..24: mol_type = other DNA
cctggtgcatcgatactctgtgct

SEQ ID NO: 47 - DNA, 27 bp, synthetic construct
  source 1..27: mol_type = other DNA
agaaaaactagatgacattgttcagca

SEQ ID NO: 48 - DNA, 27 bp, synthetic construct
  source 1..27: mol_type = other DNA
agaggtggtgaggggatgcaaagaact

SEQ ID NO: 49 - DNA, 26 bp, synthetic construct
  source 1..26: mol_type = other DNA
tgcctttggaaagtattactccaggc

SEQ ID NO: 50 - DNA, 24 bp, synthetic construct
  source 1..24: mol_type = other DNA
accagagccaatggaacttgatgg

SEQ ID NO: 51 - DNA, 27 bp, synthetic construct
  source 1..27: mol_type = other DNA
tgtgaaagagaaggaagaggggccaga

SEQ ID NO: 52 - DNA, 24 bp, synthetic construct
  source 1..24: mol_type = other DNA
atgaggcccctcaggagaggctca

SEQ ID NO: 53 - DNA, 26 bp, synthetic construct
  source 1..26: mol_type = other DNA
attgctctttcactgctttcaactgg

SEQ ID NO: 54 - DNA, 26 bp, synthetic construct
  source 1..26: mol_type = other DNA
ttgaagcactggacaatgccacatac

SEQ ID NO: 55 - DNA, 29 bp, synthetic construct
  source 1..29: mol_type = other DNA
aagagttgcgggaggctgtcggctaataa

SEQ ID NO: 56 - DNA, 27 bp, synthetic construct
  source 1..27: mol_type = other DNA
gccagacttcaagatttcagaaaaggg

SEQ ID NO: 57 - DNA, 30 bp, synthetic construct
  source 1..30: mol_type = other DNA
actaagcttgaataaaatttctgctgatac

SEQ ID NO: 58 - DNA, 28 bp, synthetic construct
  source 1..28: mol_type = other DNA
taactcgggaactgttaaccaaattatg

SEQ ID NO: 59 - DNA, 28 bp, synthetic construct
  source 1..28: mol_type = other DNA
tatttacctgaatcacatcgagccactg

SEQ ID NO: 60 - DNA, 26 bp, synthetic construct
  source 1..26: mol_type = other DNA
ccagacctttcccaaagctcaggatt

SEQ ID NO: 61 - DNA, 27 bp, synthetic construct
  source 1..27: mol_type = other DNA
ggaataagacggcaggaggaagagaga

SEQ ID NO: 62 - DNA, 30 bp, synthetic construct
  source 1..30: mol_type = other DNA
atggaagaatctcacttcaattctaacccg